(19) United States
(12) Patent Application Publication
(10) Pub. No.: US 2025/0114799 A1
(43) Pub. Date: Apr. 10, 2025

(54) Multistage Dielectrophoretic Filter System for Extracellular Vesicles

(71) Applicant: Exokeryx, Inc., Solana Beach, CA (US)

(72) Inventors: Bryan Joseph Rice, Hillsboro, OR (US); Daniel Philip Heineck, San Diego, CA (US); Michael Van Nguyen, San Diego, CA (US); Jordan Alexandra Saalfeld, San Diego, CA (US)

(21) Appl. No.: 18/910,840

(22) Filed: Oct. 9, 2024

Related U.S. Application Data
(60) Provisional application No. 63/589,128, filed on Oct. 10, 2023.

(51) Int. Cl. B03C 5/02; B03C 5/00; G01N 1/34; G01N 33/68

(52) U.S. Cl. CPC B03C 5/026; B03C 5/005; G01N 1/34; G01N 33/6896; G01N 2800/2835

(57) ABSTRACT

In one embodiment, a system includes a multistage dielectrophoretic filter system for purification of extracellular vesicles from a complex sample comprising an array of fluidic cells having electrodes that receive and process the sample, fluid transfer devices, actuated valves, storage containers, an electronic control board and power supply. In another embodiment, an extracellular vesicle analysis system for analyzing biomarkers in the purified extracellular vesicles is described. In yet another embodiment are described methods for purifying extracellular vesicles, and analyzing extracellular vesicle biomarker profiles for identifying subjects for treatment or for diagnosis.

### PRIORITY

This application claims the benefit under 35 U.S.C. § 119 (c) of U.S. Provisional Patent Application No. 63/589,128, filed 10 Oct. 2023, which is incorporated herein by reference.

### TECHNICAL FIELD

This disclosure relates generally to lab-on-chip diagnostic platforms, and in particular relates to detection of extracellular vesicle biomarkers using lab-on-a-chip diagnostics.

### BACKGROUND

There is a growing demand for diagnostic markers for early disease detection, which can increase the odds of survivability. Early disease detection requires sensitive diagnostic tools to detect low quantities of biomarkers indicative of disease. Few methods exist with the sensitivity and specificity necessary to detect diseases such as cancer and neurodegenerative diseases before considerable progression has taken place. Thus, it may be useful to develop highly sensitive lab-on-chip tests targeting the detection of early disease.

Extracellular vesicles (EVs) are membranous nanoparticles that facilitate intercellular communication via their biomolecular components (e.g., proteins, lipids, carbohydrates, and nucleic acids). EVs are dense information compartments continuously released from originating cells and contain biomarkers that mimic those of their originating cells. EVs are present in biological fluids (e.g., blood, urine, cerebrospinal fluid, etc.), and EV-associated markers can exhibit longer half-lives and increased stability than free circulating biomarkers. Thus, EVs provide an accessible source of biomarkers that are continuously released from live cells within the body.

Detection methods for cancers and neurodegenerative diseases rely upon costly, time intensive, and often invasive methods (e.g., tissue biopsy, computerized tomography, magnetic resonance imaging, endoscopy, etc.). Moreover, many of these diseases have no available tests for early detection. Currently, liquid biopsy tests rely on free circulating markers released during tumor cell death, rather than a continuous and sustained cellular process such as EV secretion. Thus, EVs represent a valuable bio-compartment for the early, minimally-invasive detection of disease-associated biomarkers from their parent cells (e.g., tumor cells, neurons affected by neurodegeneration, inflammatory cells, etc.). Existing EV-based detection methods use either laborious, “brute force” concentration methods like centrifugation and size exclusion, or rely on dilute circulating concentrations of EV, which reduces sensitivity and accuracy. Many existing EV marker analysis methods use fluorescence-based detection, which is limited in sensitivity and specificity. There is hence a need in the art for improved methods that provide a solution for efficient biomarker concentration, sensitivity, and accuracy.

### SUMMARY OF PARTICULAR EMBODIMENTS

In particular embodiments, the dielectrophoresis (DEP) filter system may be used to separate, purify, concentrate EVs from conditioned media, plasma or other bodily fluids using a chip-based electrode array positioned in a fluidic cell. In particular embodiments, the DEP is a multistage dielectrophoretic filter system comprising a plurality of housings. Each housing comprises an array of fluidic cells, each comprising an input fluid channel and an output fluid channel that is configured to receive and process a sample comprising one or more EVs for purification. Each fluidic cell also comprises a semiconductor array comprising a plurality of electrodes. A plurality of fluid transfer devices are in fluid communication between two of the array of fluidic cells via the input and output fluid channels such that the fluid transfer devices transfer the processed sample between the two fluidic cells via the input fluid channel and the output fluid channel. The system further comprises an input fluid transfer device that is in fluid communication with a first fluidic cell in the array, and an output fluid transfer device in fluid communication with an output fluid channel in a last fluidic cell in the array, one or more fluid actuators, one or more storage containers, at least one electronic control board in operable communication with the plurality of fluid transfer devices, the input fluid transfer device, and the output fluid transfer device, and a power supply in operable communication with the electronic control board.

In particular embodiments, the extracellular vesicle (EV) analysis systems disclosed herein enable improved quantitation of EV-associated biomarkers. In particular embodiments, the EV analysis system comprises a manifold, a plurality of solenoid actuated values in fluid communication with the manifold, a plurality of analyte reservoirs, one or more analyte isolation and/or tagging chambers, one or more analyte sensor chambers, and one or more fluid transfer devices. In particular embodiments, the EV analysis system further comprises a housing that houses the components described above.

In particular embodiments, there is provided a method for purifying an EV of interest. The method may begin by obtaining a biological sample comprising EVs. Next, the method may comprise applying the sample into a first fluidic cell in an array of fluidic cells of a multistage dielectrophoretic (DEP) filter system. Then the method may comprise tuning the fluidic cell using one or more capture parameters suitable for capturing the EV of interest from the sample, but not other biological particles. Then the method may comprise applying a wash solution into the fluidic cell to move the uncaptured biological particles to a waste reservoir. Then the method may comprise stopping the tuning to release the captured EV of interest. Then the method may comprise applying an elution solution to the fluidic cell. Then the method may comprise applying the eluted EV of interest to a successive fluidic cell in the array. Then the method may comprise repeating steps 1130-1170 n times, where n=total number of fluidic cells in the array-1. Finally, the method may comprise eluting the EV of interest from the last fluidic cell in the array. Particular embodiments may repeat one or more steps of the method where appropriate.

In particular embodiments, there is provided a method for determining whether a subject is at risk, or suffering from a disease or disorder that needs treatment. The method may begin by obtaining a sample comprising one or more EVs from the subject. Next the method may comprise purifying one or more EVs from the sample. Then the method may comprise determining a biomarker expression profile for the purified EVs. Then the method may comprise identifying the subject as at risk, or suffering from the disease or disorder based on the biomarker expression profile. Finally, the method may comprise comparing the biomarker expression profile with a reference database of EV biomarker expression profiles, wherein identifying the subject as at risk, or suffering from the disease is based on the comparison. Particular embodiments may repeat one or more steps of the method where appropriate.

In particular embodiments, there is provided a method for monitoring the clinical status of a disease or disorder in a subject. The method may begin by obtaining a control sample comprising one or more EVs from the subject. Second the method may comprise purifying from the control sample, EVs associated with the disease or disorder. Third the method may comprise quantifying a control expression level of one or more biomarkers associated with the disease or disorder. Fourth the method may comprise obtaining a test sample comprising one or more EVs from the subject. Fifth the method may comprise purifying from the test sample, EVs associated with the disease or disorder. Sixth the method may comprise quantifying an updated expression level of the one or more biomarkers associated with the disease or disorder, wherein, a change in updated expression level of the one or more biomarkers compared to the control expression level indicates a change in the clinical status of the disease or disorder. Then the method may comprise repeating the fourth through sixth steps. Particular embodiments may repeat one or more steps of the method where appropriate.

Certain technical challenges exist for purifying and quantitating EVs. One technical challenge may include obtaining enriched EVs of interest at a high level of recovery and purity. The solution presented by the embodiments disclosed herein to address this challenge may be the multistage dielectrophoretic (DEP) filter system discloses herein. Another technical challenge may include quantifying EV-associated biomarkers with a high degree of sensitivity and accuracy. The solution presented by the embodiments disclosed herein to address this challenge may be the extracellular vesicle (EV) analysis system disclosed herein.

Certain embodiments disclosed herein may provide one or more technical advantages. A technical advantage of the embodiments may include obtaining a recovery of EVs in a range from approximately 15% to approximately 99% compared to existing systems and methods. Particularly, one technical advantage of the embodiments may include obtaining a recovery of EVs up to approximately 80%, up to approximately 85%, up to approximately 90%, up to approximately 95%, or up to approximately 99%. Another technical advantage of the embodiments may include a recovery of EVs that is approximately 2-fold higher to approximately 10-fold higher compared to EVs in the input sample when the DEP filter system is not used. Particularly, one technical advantage of the embodiments may include a recovery of EVs that is approximately 4-fold higher compared to EVs in the input sample when the DEP filter system is not used. Yet another technical advantage of the embodiments may include an increased purity of EVs of interest ranging from approximately 15% to approximately 99% compared to existing systems and methods. Particularly, one technical advantage of the embodiments may include increasing purity up to approximately 80%, up to approximately 85%, up to approximately 90%, up to approximately 95%, or up to approximately 99%. Yet another technical advantage of the embodiments may include improved quantification of EV-associated biomarkers that is approximately 4 orders of the input sample to approximately 7 orders of the input sample compared to existing systems and methods. Certain embodiments disclosed herein may provide none, some, or all of the above technical advantages. One or more other technical advantages may be readily apparent to one skilled in the art in view of the figures, descriptions, and claims of the present disclosure.

In particular embodiments, the techniques described herein relate to a multistage dielectrophoretic (DEP) filter system for extracellular vesicles (EVs) purification, including: a plurality of housings, each housing including: an array of fluidic cells, each including an input fluid channel and an output fluid channel, configured to receive and process a sample including one or more EVs for purification, each fluidic cell including: a semiconductor array including a plurality of electrodes; a plurality of fluid transfer devices in fluid communication between two of the array of fluidic cells via the input fluid channel and the output fluid channels, wherein the fluid transfer devices transfer a processed sample between two fluidic cells via the input fluid channel and the output fluid channel; an input fluid transfer device in fluid communication with a first fluidic cell in the array, and an output fluid transfer device in fluid communication with an output fluid channel in a last fluidic cell in the array; one or more fluid actuators; one or more storage containers; at least one electronic control board in operable communication with the plurality of fluid transfer devices, the input fluid transfer device, and the output fluid transfer device; and a power supply in operable communication with the electronic control board.

In particular embodiments, the techniques described herein relate to a multistage DEP filter system, further including a computer in operable communication with the electronic control board, said computer including: at least one processor; at least one memory; an I/O interface; a display; at least one communication interface; and a library of algorithms tangibly stored in the memory and executable by the processor, said algorithms including processor-executable instructions to actuate one or more of the plurality of fluid transfer devices, the input fluid transfer device, and the output fluid transfer device.

In particular embodiments, the techniques described herein relate to a multistage DEP filter system, wherein the semiconductor array in the fluidic cell enables operation of the multistage DEP filter system at an operating mode for EV purification that is selected from one or more of, high capture efficiency, purification, or band-pass.

In particular embodiments, the techniques described herein relate to a multistage DEP filter system, wherein the band-pass is based on size of the EV.

In particular embodiments, the techniques described herein relate to a multistage DEP filter system, wherein the electrodes include positive electrodes and negative electrodes.

In particular embodiments, the techniques described herein relate to a multistage DEP filter system, wherein the semiconductor array includes electrodes disposed in one or more electrode configurations, wherein the configuration enable generation of an electric field for capturing the EVs in the sample.

In particular embodiments, the techniques described herein relate to a multistage DEP filter system, wherein one or more of the plurality of fluidic cells have an identical electrode configuration (symmetric electrode configuration).

In particular embodiments, the techniques described herein relate to a multistage DEP filter system, wherein one or more of the plurality of fluidic cells have a non-identical electrode configuration (asymmetric electrode configuration).

In particular embodiments, the techniques described herein relate to a multistage DEP filter system, wherein the electrodes are disposed in an interdigitated electrode (IDE) geometrical pattern.

In particular embodiments, the techniques described herein relate to a multistage DEP filter system, wherein the system is constructed out of organic thin films.

In particular embodiments, the techniques described herein relate to a multistage DEP filter system, wherein one or more of the fluidic cell is coated with an anti-biofouling agent.

In particular embodiments, the techniques described herein relate to a multistage DEP filter system, wherein the fluidics cell is tunable for one or more EV capture parameters.

In particular embodiments, the techniques described herein relate to a multistage DEP filter system, wherein the capture parameters are selected from the group consisting of voltage, frequency, field source geometry, fluid medium, solution conductivity, surface coating, electrode pitch and electrode geometry, or a combination thereof.

In particular embodiments, the techniques described herein relate to a multistage DEP filter system, wherein the fluid transfer device includes: a syringe; an actuator in operable communication with the syringe; a valve in operable communication with the actuator; and a motor in operable communication with the valve.

In particular embodiments, the techniques described herein relate to a multistage DEP filter system, wherein the valve is a solenoid valve.

In particular embodiments, the techniques described herein relate to an extracellular vesicle (EV) analysis system, including: a manifold; a plurality of solenoid actuated valves in fluid communication with the manifold; a plurality of analyte reservoirs; one or more analyte isolation and/or tagging chambers; one or more analyte sensor chambers; and one or more fluid transfer devices.

In particular embodiments, the techniques described herein relate to an analysis system, further including a housing that houses the multistage DEP filter system.

In particular embodiments, the techniques described herein relate to an analysis system, wherein the fluid transfer device is a syringe.

In particular embodiments, the techniques described herein relate to an analysis system, wherein the fluid transfer device includes: a syringe; an actuator in operable communication with the syringe; a valve in operable communication with the actuator; and a motor in operable communication with the valve.

In particular embodiments, the techniques described herein relate to an analysis system, wherein the valve is a solenoid valve.

In particular embodiments, the techniques described herein relate to a system including: the multistage dielectrophoretic (DEP) filter system; the EV analysis system; and a computer in operable communication with the multistage dielectrophoretic (DEP) filter system and the detection system, said computer including: at least one processor; at least one memory; an I/O interface; a display; at least one communication interface; and a library of algorithms tangibly stored in the memory and executable by the processor, said algorithms including processor-executable instructions to operate the multistage dielectrophoretic (DEP) filter system and the detection system.

In particular embodiments, the techniques described herein relate to a method for purifying an EV of interest including: (a) obtaining a biological sample including EVs; (b) applying the sample into a first fluidic cell in an array of fluidic cells of a multistage dielectrophoretic (DEP) filter system; (c) tuning the fluidic cell using one or more capture parameters suitable for capturing the EV of interest from the sample, but not other biological particles; (d) applying a wash solution into the fluidic cell to move the uncaptured biological particles to a waste reservoir; (e) stopping the tuning to release the captured EV of interest; (f) applying an elution solution to the fluidic cell to clute the EV; (g) applying the eluted EV of interest to a successive fluidic cell in the array; (h) repeating (c)-(g) n times, where n=total number of fluidic cells in the array-1; and (i) eluting the EV of interest from the last fluidic cell in the array.

In particular embodiments, the techniques described herein relate to a method, further including analyzing the eluted EV.

In particular embodiments, the techniques described herein relate to a method, wherein the analyzing is qualitative and/or quantitative.

In particular embodiments, the techniques described herein relate to a method, wherein the eluted EV is analyzed using immunofluorescence, western blotting, hybridization, PCR, mass spectrometry, bioanalyzer, nucleic acid sequencing, silver staining, single particle interferometric reflectance imaging sensor (SP-IRIS), or a combination thereof.

In particular embodiments, the techniques described herein relate to a method, wherein the eluted EV is analyzed using an extracellular vesicle (EV) analysis system.

In particular embodiments, the techniques described herein relate to a method, wherein the eluted EV is analyzed using the extracellular vesicle (EV) analysis system.

In particular embodiments, the techniques described herein relate to a method, further including concentrating the eluted EV.

In particular embodiments, the techniques described herein relate to a method, further including storing the eluted EV.

In particular embodiments, the techniques described herein relate to a method, wherein the EV is purified using the multistage dielectrophoretic (DEP) filter system.

In particular embodiments, the techniques described herein relate to a method, wherein the applying steps are performed using a fluid transfer device selected from a syringe, a pump, and an automated liquid controller.

In particular embodiments, the techniques described herein relate to a method, wherein the fluid transfer device is a syringe.

In particular embodiments, the techniques described herein relate to a method, wherein the fluid transfer device includes: a syringe; an actuator in operable communication with the syringe; a valve in operable communication with the actuator; and a motor in operable communication with the valve.

In particular embodiments, the techniques described herein relate to a method, wherein the valve is a solenoid valve.

In particular embodiments, the techniques described herein relate to a method, wherein the capture parameters are selected from the group consisting of voltage, frequency, field source geometry, fluid medium, solution conductivity, surface coating, electrode pitch and electrode geometry, and a combination thereof.

In particular embodiments, the techniques described herein relate to a method, wherein the biological sample is plasma, blood, a liquid biopsy, a cell lysate, or a tissue lysate.

In particular embodiments, the techniques described herein relate to a method of determining whether a subject is at risk, or suffering from a disease or disorder that needs treatment including: obtaining a sample including one or more EVs from the subject; purifying one or more EVs from the sample; determining a biomarker expression profile for the purified EVs; and identifying the subject as at risk, or suffering from the disease or disorder based on the biomarker expression profile.

In particular embodiments, the techniques described herein relate to a method, further including comparing the biomarker expression profile with a reference database of EV biomarker expression profiles, wherein identifying the subject as at risk, or suffering from the disease is based on the comparing.

In particular embodiments, the techniques described herein relate to a method, further including treating the subject.

In particular embodiments, the techniques described herein relate to a method, wherein the disease or disorder is selected from the group consisting of a cancer, an autoimmune disease, a vascular disease a neurodegenerative disease, a metabolic disease, a renal disease, and an inflammatory bowel disease (IBD).

In particular embodiments, the techniques described herein relate to a method, wherein the EVs are purified using the system.

In particular embodiments, the techniques described herein relate to a method, wherein the EVs are purified using the method.

In particular embodiments, the techniques described herein relate to a method, wherein the biomarker expression profile is determined using immunofluorescence, western blotting, hybridization, PCR, mass spectrometry, bioanalyzer, nucleic acid sequencing, silver staining, single particle interferometric reflectance imaging sensor (SP-IRIS), or a combination thereof.

In particular embodiments, the techniques described herein relate to a method for monitoring a clinical status of a disease or disorder in a subject including: (a) obtaining a control sample including one or more EVs from the subject; (b) purifying from the control sample, EVs associated with the disease or disorder; (c) quantifying a control expression level of one or more biomarkers associated with the disease or disorder; (d) obtaining a test sample including one or more EVs from the subject; (e) purifying from the test sample, EVs associated with the disease or disorder; and (f) quantifying an updated expression level of the one or more biomarkers associated with the disease or disorder, wherein, a change in updated expression level of the one or more biomarkers compared to the control expression level indicates a change in the clinical status of the disease or disorder.

In particular embodiments, the techniques described herein relate to a method, further including repeating steps (d)-(f).

In particular embodiments, the techniques described herein relate to a method, wherein the disease or disorder is selected from the group consisting of a cancer, an autoimmune disease, a vascular disease, a neurodegenerative disease, a metabolic disease, a renal disease, and an inflammatory bowel disease (IBD).

In particular embodiments, the techniques described herein relate to a method, wherein the EVs are purified using the system.

In particular embodiments, the techniques described herein relate to a method, wherein the EVs are purified using the method.

In particular embodiments, the techniques described herein relate to a method, wherein the quantifying is by immunofluorescence, western blotting, hybridization, PCR, mass spectrometry, bioanalyzer, nucleic acid sequencing, silver staining, single particle interferometric reflectance imaging sensor (SP-IRIS), or a combination thereof.

In particular embodiments, the techniques described herein relate to a method, wherein the subject is treated for the disease or disorder before step (d).

In particular embodiments, the techniques described herein relate to a method, wherein: a lower updated expression level of the one or more biomarkers compared to the control expression level indicates that the subject is in remission and/or responding to treatment; a lower updated expression level of the one or more biomarkers compared to the control expression level indicates that the subject is in relapse and/or not responding to treatment; a higher updated expression level of the one or more biomarkers compared to the control expression level indicates that the subject is in remission and/or responding to treatment; or a higher updated expression level of the one or more biomarkers compared to the control expression level indicates that the subject is in relapse and/or not responding to treatment.

The embodiments disclosed herein are only examples, and the scope of this disclosure is not limited to them. Particular embodiments may include all, some, or none of the components, elements, features, functions, operations, or steps of the embodiments disclosed herein. Embodiments according to the invention are in particular disclosed in the attached claims directed to a method, a storage medium, a system and a computer program product, wherein any feature mentioned in one claim category, e.g. method, can be claimed in another claim category, e.g. system, as well. The dependencies or references back in the attached claims are chosen for formal reasons only. However any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

### BRIEF DESCRIPTION OF THE DRAWINGS

FIG. 1 illustrates an example diagram of a cross-section view of a dielectrophoretic concept.

FIGS. 2A and 2B show top and bottom views of an exemplary multistage dielectrophoretic (DEP) filter system. FIG. 2A illustrates an example diagram of a top-view of a multistage DEP filter system FIG. 2B illustrates an example diagram of a bottom-view of a multistage dielectrophoretic filter system.

FIGS. 3A and 3B is an illustration of an example method and example schematic flowchart for purifying EVs of interest. FIG. 3A is an example method for purifying an EV of interest. FIG. 3B illustrates an example flowchart of a multistage dielectrophoretic filter system.

FIG. 4 illustrates an example flowchart of multistage dielectrophoretic filter system.

FIGS. 5A and 5B illustrate exemplary system comprising the DEP filter system and the EV analysis system (detection system, detection apparatus). FIG. 5A illustrates an example isometric diagram of a generalized fluidics delivery approach of a detection apparatus. FIG. 5B illustrates an example isometric diagram of a generalized fluidics delivery approach.

FIG. 6 illustrates an example diagram of a complementary metal-oxide semiconductor array in which one embodiment may operate.

FIGS. 7A and 7B illustrate exemplary electrode configuration and geometry. FIG. 7A illustrates an example diagram of an electrode geometry concept. FIG. 7B illustrates an example diagram of an electrode configuration concept.

FIG. 8 illustrates an example method of determining whether a subject is at risk, or suffering from a disease or disorder that needs treatment.

FIG. 9 illustrates an example method for monitoring the clinical status of a disease or disorder in a subject.

FIG. 10 illustrates a diagram of an example computer system.

FIG. 11 illustrates an example comparison of EV recovery based on biomarker signal in an input sample before not subject to the DEP filtration system and the output sample after purification using the DEP filtration system disclosed herein.

FIG. 12 is an example illustration of qualitative purity indicators between the input and output samples from FIG. 11 using CD9 as the desired biomarker of interest and fibronectin as the undesired impurity.

FIGS. 13A and 13B show a comparison of biomarker quantification using industry standard methods and the EV analysis and quantification system and methods disclosed herein. FIG. 13A is an example illustration of industry standard biomarker quantification using a fluorescence-based approach, which is limited to a dynamic range of about 2 orders of input sample concentration. FIG. 13B is an example illustration of biomarker quantification using the EV analysis systems and methods disclosed herein, which yields over 4.5 orders of input sample concentration, theoretically extending to 7 orders.

### DESCRIPTION OF EXAMPLE EMBODIMENTS

Researchers and clinicians require access to highly concentrated and purified extracellular vesicles (EVs) from biological samples such as conditioned media, plasma, urine, cerebral spinal fluid, saliva, tear(s), and any other suitable biological media to investigate intercellular communication, disease genesis and progression, and the efficacy of various therapies. As used herein, “extracellular vesicles (EVs)” refers to lipid membrane-bound bio-compartments derived from cells, or derived synthetically. Non-limiting examples of EVs include exosomes, microvesicles, apoptotic bodies, and macrovesicles, exosomes, microvesicles, apoptotic bodies, macrovesicles, endosomes, vacuoles, lysosomes, secretory vesicles, transport vesicles, peroxisomes, phagosomes, or fragments thereof. The information contained within EVs, in the form of biomarkers (e.g., proteins, lipids, carbohydrates, and nucleic acids, etc.), found both on the surface of the membrane and inside the vesicle, may be of significant value to those who wish to diagnose and characterize diseases. Currently, EV isolation techniques require the researcher or clinician to choose between high recovery (e.g., capturing most of the EVs at the expense of also retaining many impurities) or high purity (e.g., at the expense of losing 99% or more of the EVs in the sample). Certain technical challenges exist for purifying and quantitating EVs. One technical challenge may include obtaining enriched EVs of interest at a high level of recovery and purity. The solution presented by the embodiments disclosed herein to address this challenge may be the multistage dielectrophoretic (DEP) filter system discloses herein. Another technical challenge may include quantifying EV-associated biomarkers with a high degree of sensitivity and accuracy. The solution presented by the embodiments disclosed herein to address this challenge may be the extracellular vesicle (EV) analysis system disclosed herein. The multistage dielectrophoretic filter system and methods described herein overcomes the disadvantages associated with current approaches.

The subject matter of the present disclosure is described with reference to the figures. It should be understood that numerous specific details, relationships, and methods are set forth in this Description and accompanying Figures to provide a more complete understanding of the subject matter disclosed herein. For purposes of clarity of disclosure and not by way of limitation, the detailed description is divided into the following subsections:
1. Definitions
2. Dielectrophoretic (DEP) filter systems
3. Methods of purifying EVs
4. Extracellular vesicle (EV) analysis systems
5. Integrated Systems for EV purification and Analysis
6. Methods of Diagnosis
7. Systems and Methods
8. Examples
9. Recitation of Embodiments
10. Miscellaneous

### 1. Definitions

As used herein, the singular form “a”, “an” and “the” include plural referents unless the context clearly dictates otherwise.

The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

As used herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

As used herein, “automatically” and its derivatives means “without human intervention,” unless expressly indicated otherwise or indicated otherwise by context.

As used herein, the term “extracellular vesicles (EVs)” includes exosomes, microvesicles, apoptotic bodies, macrovesicles, endosomes, vacuoles, lysosomes, secretory vesicles, transport vesicles, peroxisomes, phagosomes, and fragments thereof.

As used herein, the terms “chamber”, “fluid cell”, “fluidic cell” are used interchangeably with reference to a compartment with a cavity/volume having an electrode array on a bottom surface, adhesive sides, an input fluid channel (inlet fluid channel), an output fluid channel (outlet fluid channel) and a top cover for enclosing the various elements of the compartment including a sample that would pass through the chamber/fluid cell/fluidic cell during operation of the dielectrophoresis (DEP) filter system.

As used herein, the terms “filter”, “separate”, “purify” and various iterations thereof including “filtration”, “separation”, “purification” are used synonymously in reference to isolating a desired population of extracellular vesicles (EVs) from a complex biological sample comprising EVs, proteins, carbohydrates, lipids, salts, small compounds, aggregates and cells.

As used herein, the terms “reservoir”, “storage tube”, “storage container”, “consumable cartridge for reagents” are used interchangeably and refer to a means for storing reagents including wash buffers that are delivered to the DEP filter system during EV purification.

### 2. Dielectrophoretic (DEP) Filter Systems

To interrogate the contents and composition of EVs, it is advantageous to have a method to isolate them from biologically relevant fluids at a high recovery percentage and purity. Dielectrophoresis (DEP) may be used to separate, purify, concentrate EVs from conditioned media, plasma or other bodily fluids using a chip-based electrode array positioned in a fluidic cell. In particular embodiments, a multistage dielectrophoretic filter system may streamline the process of isolating and recovering EVs, greatly reducing the time and number of sample manipulations required for filtering and purifying EVs.

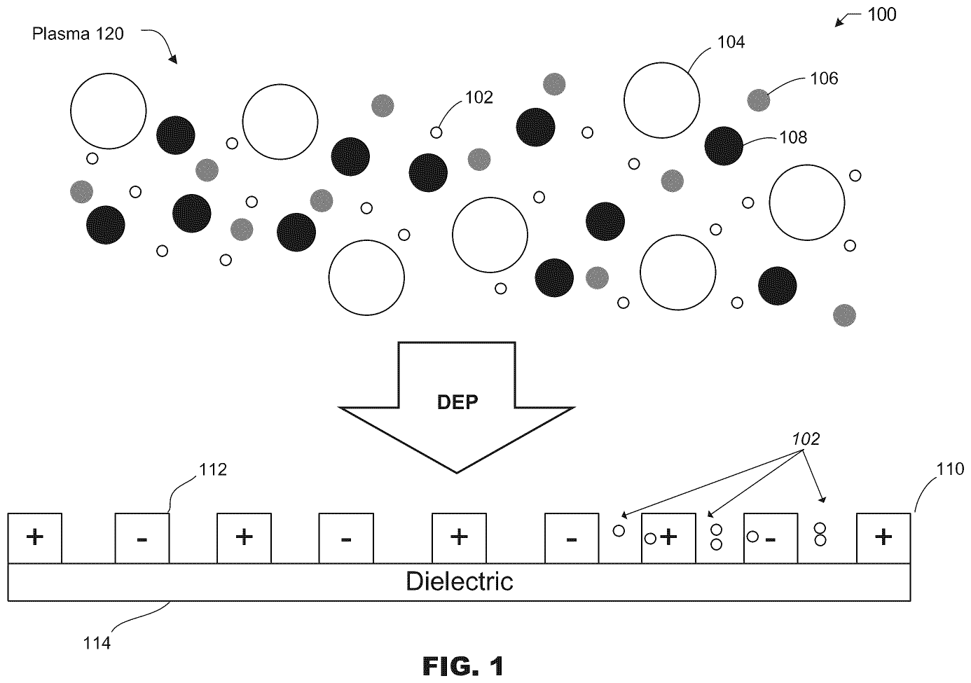

FIG. 1 illustrates an example diagram 100 of a cross-section view of a dielectrophoretic (DEP) concept. In particular embodiments, a biological sample (e.g., plasma 120) may be loaded onto an electrode array, wherein extracellular vesicles (EVs) 102 may be concentrated and/or isolated by a detection apparatus via one or more multistage dielectrophoretic filter systems. EVs 102 are lipid membrane-bound bio-compartments derived from cells and hold information in the form of biomarkers found both on the surface of the membrane and inside the vesicle, which may be of value in diagnosing and characterizing disease. In particular embodiments, to interrogate the contents and compositions of one or more EVs 102, the EVs 102 may be isolated from the biological sample (e.g., plasma 120). To isolate the one or more EVs 102 at a high recovery percentage and purity from plasma 120 and/or any other suitable biological sample, DEP may be applied to and/or used on the biological sample.

DEP is a technique that uses the net force that acts on particles with an asymmetric polarizability in the presence of a radio frequency (RF) field. As demonstrated by diagram 100, plasma 120 of the biological sample may enter a DEP chamber, as described in later figures. As an example and not by way of limitation, plasma 120 may consist of EVs 102 as well as various other biological particles 104, 106, and 108. As way of example and not by limitation, DEP may be used to filter EV 102 nanoparticles from plasma 120 and/or other biological sample using a chip-based electrode array positioned in a fluidic cell.

In particular embodiments, a dielectrophoretic force may be applied to a biological fluid (e.g., plasma 120), wherein one or more particles (e.g., EV 102) may polarize in a particular way that differs from the surrounding medium in the presence of an electric field gradient. As an example and not by way of limitation, the gradient may be tuned in strength and direction based on device geometry, thus allowing for one to concentrate or deplete EVs at a high-field gradient region such as an edge.

EVs 102 may be measured as spherical objects of roughly 30-10,000 nm. As an example and not by way of limitation, for any given combination of voltage, frequency, electrode pitch, and/or electrode geometry, a subrange of this set of particles may be net attracted to the one or more electrodes. This phenomenon may be referred to as positive DEP. Particles experiencing positive DEP will henceforth be referred to as the band of attraction. As used herein, “band of attraction” may refer to a subrange of EV 102 sizes. For the avoidance of doubt, it is clear that the band of attraction is a tunable range of EV 102 sizes.

Combinatorial detection of the presence of multiple EVs 102, along with analysis with one or more machine learning (ML) models, algorithms, and/or modules, may be useful for sensitive and specific diagnosis of early cancer and other diseases from biological fluids.

In particular embodiments, the DEP is a multistage dielectrophoretic filter system comprising a plurality of housings, each housing comprising an array of fluidic cells, each comprising an input fluid channel and an output fluid channel and configured to receive and process a sample comprising one or more EVs for purification, each fluidic cell comprising a semiconductor array comprising a plurality of electrodes; a plurality of fluid transfer devices in fluid communication between two of the array of fluidic cells via the input fluid channel and output fluid channel, wherein the fluid transfer devices transfer the processed sample between the two fluidic cells via the input fluid channel and the output fluid channel; an input fluid transfer device in fluid communication with a first fluidic cell in the array, and an output fluid transfer device in fluid communication with an output fluid channel in a last fluidic cell in the array; one or more fluid actuators; one or more storage containers; at least one electronic control board in operable communication with the plurality of fluid transfer devices, the input fluid transfer device, and the output fluid transfer device; and a power supply in operable communication with the electronic control board.

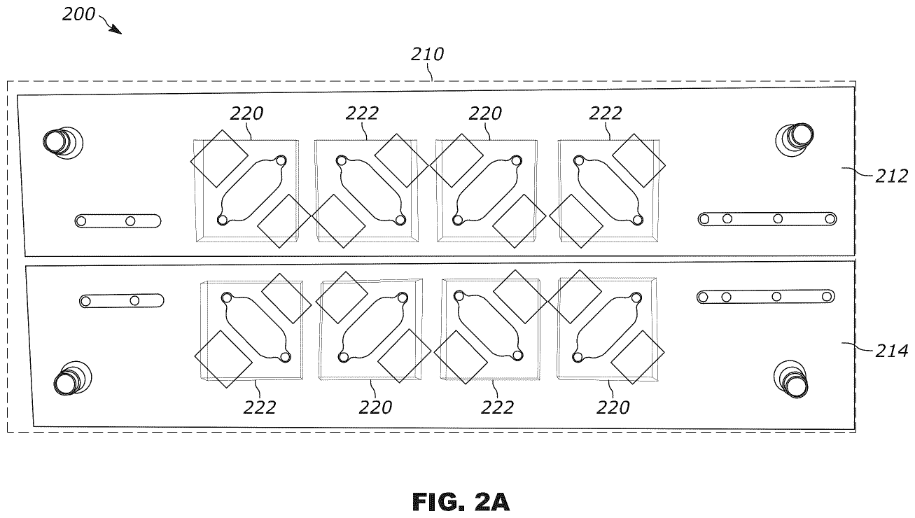

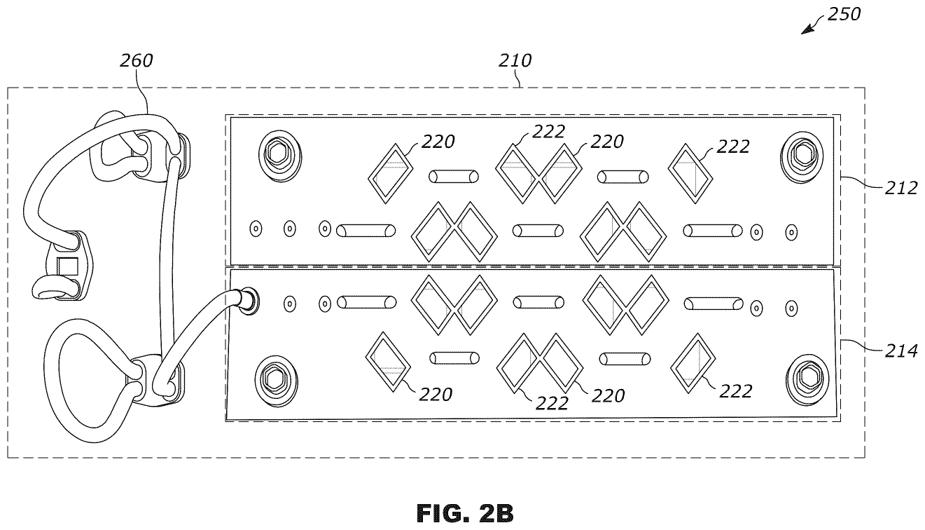

FIG. 2A illustrates an example diagram 200 of a top-view of a multistage dielectrophoretic filter system 210. In particular embodiments, the multistage dielectrophoretic filter system 210 may be programmed to filter EVs 102 between 30 nm and 10,000 nm. In particular embodiments, a multistage dielectrophoretic filter system 210 may include one or more physical housings 212, 214, one or more electronic boards, one or more DEP stages (e.g., chambers, fluidic cells) with electrode configurations (e.g., electrode configurations 220, 222), fluidics machinery (e.g., fluid transfer devices), one or more reservoirs for reagents, reservoirs containing the multistage DEP filter system, and one or more mechanisms (e.g., syringes, pumps, automated liquid controllers) for accepting sample inputs and/or flowing sample outputs (e.g., eluates) to one or more containers for storage, shipment, and/or subsequent analysis. As used herein, electrode configurations 220, 222 may refer to configurations of electrode arrays. Although FIG. 2A discloses a particular electrode configuration 220, 222, this disclosure contemplates that a multistage dielectrophoretic filter system 210 may contain any suitable number or configuration of electrodes and/or electrode arrays.

In particular embodiments, the multistage dielectrophoretic filter system 210 may contain two or more physical housings 212, 214, wherein one or more physical housings 212, 214 may receive input biological samples (e.g., plasma 120), and one or more physical housings 212, 214 may output the biological sample into one or more containers for storage, shipment, and/or subsequent analysis. In particular embodiments, the multistage dielectrophoretic filter system 210 may fit within an extracellular vesicle isolation product.

In particular embodiments, each stage of the multistage dielectrophoretic filter system 210 may be tuned with one or more particular sets of operating conditions. In particular embodiments, the semiconductor array in the fluidic cell enables operation of the multistage DEP filter system at an operating mode for EV purification that is selected from one or more of, high capture efficiency, purification, or band-pass. As an example and not by way of limitation, each DEP stage may be enabled to operate at a particular operating mode, such as high capture efficiency, purification, or size-based band-pass filtering. For example, each DEP stage (chamber, fluidic cell) may operate at a particular operating mode, in series with other DEP stages, without creating interference (e.g., electromagnetic interference). As another example and not by way of limitation, a first DEP stage may operate as a high capture efficiency filter system, followed by a second DEP stage operating as a purification filter system, wherein the combination of the systems may yield a high recovery, high purity output.

In particular embodiments, one or more EVs 102 containing biological fluids may be passed over one or more electrodes of one or more electrode configurations 220, 222, wherein the electrodes generate an electric field, resulting in the capture of one or more EVs 102. In particular embodiments, one or more filters of the multistage dielectrophoretic filter system 210 may comprise an alternating polarity electrode geometry with electroformed or sputtered electrodes with a pitch between 100 nm and 200 um. In particular embodiments, the one or more electrodes of the one or more electrode configurations 220, 222 may operate at a frequency between 100 Hz and 5 MHz and voltage between 0.1 V and 20 V in the biological sample (e.g., plasma, urine, cerebral spinal fluid or any other prepared buffer), with a conductivity between 0.1 mS and 30 mS.

In particular embodiments, when an alternating current (AC) waveform is run through positive electrodes 110 and/or negative electrodes 112, the one or more EVs may experience an attractive force that is a function of the RF, voltage, plasma conductivity, EV particle size, and/or EV particle charge. In this example, positive electrodes 110 and/or negative electrodes 112 may be constructed by metal or any other suitable material. In particular embodiments, the positive electrodes 110 and/or negative electrodes 112 may be separated by a dielectric material 114. In particular embodiments, EVs 102 may be attracted to one or more of the positive electrodes 110 and/or negative electrodes 112 on the surface of the dielectric material 114.

In particular embodiments, the semiconductor array comprises electrodes disposed in one or more electrode configurations 220, 222, wherein the configuration enables generation of an electric field for capturing the EVs in the sample.

In particular embodiments, one or more of the plurality of fluidic cells have an identical electrode configuration (symmetric electrode configuration). Use of identical electrode configurations in multiple fluidic cells enables obtaining EVs that have successively higher purity compared to purity achieved from a previous fluidic cell.

In particular embodiments, one or more of the plurality of fluidic cells have a non-identical electrode configuration (asymmetric electrode configuration). In particular embodiments, the electrodes are disposed in an interdigitated electrode (IDE) geometrical pattern. As an example and not by way of limitation, the IDE geometric pattern may be in the form of parallel lines, or parallel saw-tooth patterns. Combinations of various geometric patterns for the electrodes may also be used.

In particular embodiments, when an alternating current (AC) waveform is run through positive electrodes 110 and negative electrodes 112, the one or more EVs 102 may experience an attractive force that is a function of the RF, voltage, plasma conductivity, EV particle size, and EV particle charge. In this example, the positive electrodes 110 and/or negative electrodes 112 may be constructed by platinum or any other suitable material. In particular embodiments, the positive electrodes 110 and/or negative electrodes 112 may be separated by dielectric material.

In particular embodiments, a fluidic chamber housing the one or more electrodes of one or more electrode configurations 220, 222 may be coated with an anti-biofouling thin film. As an example and not by way of limitation, the anti-biofouling thin film may be of an organic composition with a particular hydrophilicity, porosity, and/or thickness. As another example and not by way of limitation, the anti-biofouling thin film may be of an inorganic composition with a particular hydrophilicity, porosity, and/or thickness. As an example and not by way of limitation, bio-fouling agents in the anti-biofouling thin film may include methylacrylate, tributyltin (TBT), triphenyltin (TPT) and polyethylene glycol (PEG). For example, in particular non-limiting embodiments, the anti-biofouling thin film is a methyl acrylate polymer film. In particular embodiments, different fluid cells may be coated with anti-biofouling thin films comprising different bio-fouling agents to getter different contaminants. In particular embodiments, a fluid cell may be coated with anti-biofouling thin films comprising a combination of bio-fouling agents to getter a combination of contaminants. Anti-biofouling agents and films comprising anti-biofouling agents are well known and one of skill in the art would be readily able to select a suitable anti-biofouling thin film for use with the DEP system described herein.

In particular embodiments, the multistage dielectrophoretic filter system 210 may include one or more fluidics systems, wherein the one or more fluidics systems may move fluid (e.g., biological sample, wash volume, etc.) between one or more stages of the multistage dielectrophoretic filter system 210 and/or between filter systems. In particular embodiments, the one or more fluidics systems may move operate as an immediate storage and/or waste system. In particular embodiments, the one or more fluidics systems may recover the output of any given DEP stage of the multistage dielectrophoretic filter system 210.

In particular embodiments, the multistage dielectrophoretic filter system 210 may contain one or more high-pass filter systems. As an example and not by way of limitation, a first high-pass filter system may be in series with one or more secondary high-pass filter systems, wherein the recoverable loss volume of a second high-pass filter system may become the band-pass eluate.

In particular embodiments, the multistage dielectrophoretic filter system 210 may include a serial configuration of two or more filter systems, wherein the captured material of the final filter system may become the eluate.

In particular embodiments, the multistage dielectrophoretic filter system 210 may include a serial configuration of two or more (fluidic cells), wherein the recoverable loss volume of the final (fluidic cell) may become the eluate.

As an example and not by way of limitation, a remaining fraction of EVs 102 may be carried away in wash volume. The wash volume may then be directed through one or more additional filters (“stages”), wherein EVs in the wash volume may have additional opportunities to recaptured and retained.

In particular embodiments, the DEP system is used to separate, purify, and concentrate the desired EVs from undesired particulates through the use of buffers to wash unwanted particulates and media downstream from the fluidic cell with the EVs 102 held stationary via DEP. As an example and not by way of limitation, the wash buffers are buffered saline solutions that are biologically safe and compatible with biological systems.

In particular embodiments, after washing, the desired captured material may be “released” upon deactivating the DEP field and the resulting eluate collected for analysis. Methods of EV isolation from biologically-relevant fluids (e.g., plasma 120) offer a higher yield and cleaner eluate than conventional methods, such as ultracentrifugation, size exclusion chromatography, and/or other conventional methods. Thus, a tunable, multistage DEP filter system to increase overall EV capture efficiency and purity in a tunable particle size band is beneficial over currently used methods.

In particular embodiments, the DEP system is used to capture the undesired particulates such that the desired EVs pass through the fluidic cell without being captured. The desired EV's are then collected for analysis. In particular embodiments, after collection of the desired EVs, the undesired particulates may be “released” by deactivating the DEP field and using wash buffers to clear the fluidic cell of any impurities. Methods of EV isolation from biologically-relevant fluids (e.g., plasma 120) offer a higher yield and cleaner eluate than conventional methods, such as ultracentrifugation, size exclusion chromatography, and/or other conventional methods. Thus, a tunable, multistage DEP filter system to increase overall EV capture efficiency and purity in a tunable particle size band is beneficial over currently used methods.

In particular embodiments, a tunable, multistage DEP filtration approach may increase overall EV capture and purity. As an example and not by way of limitation, tunable DEP filter and/or DEP capture parameters may include voltage, frequency, field source geometry (e.g., electrode and insulator pattern), fluid medium, and electrode pitch. In particular embodiments, a single pass of EV-containing plasma (e.g., plasma 120) over an electric field may be subject to some amount of EVs 102 captured by the electrodes (e.g., positive electrode 110, negative electrode 112), wherein the remaining faction of EVs may be lost to subsequent washing. As used herein, “washing” refers to a process in which the assay surface may be washed with saline or any other suitable solvent via needle, syringe, and/or any other suitable means of delivering solvent. As an example and not by way of limitation, EVs 102 lost in the washing may occur due to the force of the wash surmounting the DEP force on the one or more EVs 102 experiencing a weaker field due to being positioned further away from an electrode edge. With the addition of subsequent DEP electrode arrays, one or more EVs 102 not captured by a first array may have additional opportunities to be recaptured and retained in the elute while passing one or more subsequent electrode arrays (e.g., electrode configurations 220, 222).

In particular embodiments, surface coatings may be chosen and modified to set layer thickness conformation, porosity, and/or hydrophilicity.

In particular embodiments, DEP filters at each stage may be programmed independently in a variety of configurations. As an example and not by way of limitation, one or more filters (e.g., DEP stages) may be tuned with the same parameters and coatings to improve EV capture fraction at each filter stage, resulting in an overall high percent recovery. As another example and not by way of limitation, the one or more DEP filters may be programmed to act as a band-pass filter for EVs of a certain size range (e.g., between 30 nm and 10,000 nm) by excluding smaller nanoparticles in the first stage using one set DEP parameters and repelling large particles in a later state with different DEP parameters. As yet another example and not by way of limitation, the filters may be modified for purity, wherein each stage may sequentially purify the sample against one or more targeted impurities.

Capture and loss fractions for any singular stage of a multistage filtration system may be defined as follows:

$$( Equation l )$$

$$f c = C / T$$

$${f}_{R} = R / T$$

$${f}_{L} = L / T$$

As shown above in Equation 1 and assuming that the amount of material in a particle size and charge distribution capture range of interest (henceforth referred to as “material”) in a sample volume, the total amount of material T may be defined as T=C+R+L, where C represents the amount of captured material in the first stage, R is the amount of material washed from the first stage but that is recoverable, and L is the amount of material lost and unrecoverable even in subsequent filters. Based on the forementioned definitions, a closed form may be constructed to describe capture efficiency f_{Cn} of the n^{th} stage filter in a multistage filter system, as well as the overall capture efficiency, F, of the entire multistage filter system. As an example and not by way of limitation, a simplifying assumption that the conditions among all filters are identical may yield the following:

$${f}_{Cn} = {f}_{C} {f}_{R}^{n - 1} \qquad ( Equation 2 )$$

$$and$$

$$F = {\sum }_{i = 1}^{n} ( {f}_{Ci} ) \qquad ( Equation 3 )$$

As shown and described by capture efficiency f_{Cn} of the n^{th} stage filter in a multistage filter system in Equation 2 and the overall capture efficiency, F, of the entire multistage filter system in Equation 3, capture efficiency of a multistage system may be estimated to determine the number of stages required to obtain a desired efficiency. For example, the system with observed material values as in Table 1 (shown below) may yield the results of Table 2 (shown below).

TABLE 1
OBSERVED MATERIAL VALUES

| | |
|---|---|
| T | 2 |
| C | 0.8 |
| R | 1.1 |
| L | 0.1 |

TABLE 2
CAPTURE EFFICIENCY

| | |
|---|---|
| f_{C} | 0.4 |
| f_{R} | 0.55 |
| f_{L} | 0.05 |

In particular embodiments, a 1, 2, 3, or 4 stage filter system may ultimately yield overall system filter efficiencies F as demonstrated in Table 3, below.

TABLE 3
OVERALL SYSTEM FILTER EFFICIENCIES

| Stage n | f_{Cn} | F |
|---|---|---|
| 1 | 0.4 | 0.4 |
| 2 | 0.22 | 0.62 |
| 3 | 0.121 | 0.741 |
| 4 | 0.06655 | 0.80755 |

As shown above, a stage 2 filter may yield a capture efficiency f_{Cn} of 0.22, wherein the overall system filter efficiency F is 0.62.

In particular embodiments, a “symmetric filter” approach may be used. As used herein, a “symmetric filter” approach is one in which all stages are equivalent. In particular embodiments, the symmetric filter approach may improve upon the single filter efficiency, permitting a higher recovery rate of EVs 102. Single filter efficiency and a high EV recovery rate are valuable as some sample volumes may be critically limited (e.g., biorepository samples) so loss minimization is important. In particular embodiments, two or more filters may be tuned with the same parameters and surface coatings to improve V capture fraction at each filter stage, resulting in a high percent recovery of the overall system.

In particular embodiments, the multistage dielectrophoretic filter system 210 may be employed in an asymmetric fashion. As used herein, an “asymmetric filter” approach is one in which one or more stages may not be equivalent. As an example and not by way of limitation, DEP may be tuned via parameters such as frequency, voltage, solution conductivity, electrode geometry, surface coatings, and/or any other suitable parameter to attract particles of a particular range and repel particles outside of the particular range. In this example, each filter may be tuned to one or more different parameters and/or parameter levels. In particular embodiments, two or more successive filters may be tuned to reject particles outside of a predetermined range. By tuning two or more successive filters to reject particles outside of a predetermined range, a band-pass filter on particle size may be constructed. It is understood that the two or more successive filters may be in a symmetric or asymmetric configuration. In particular embodiments, a two-section band-pass filter system for extracellular vesicles may be constructed wherein each section of the filter system may contain one or more filter stages. As an example and not by way of limitation, the first filter system may capture all particles below a cutoff size Supper.

In particular embodiments, after purification processes have been performed, captured material may be released into the solution and transported from the first fluidic cell to the second fluidic cell. As an example and not by way of limitation, in the second fluidic cell, an additional set of DEP parameters may be utilized to capture particles below a second particle size cutoff, S_{lower}. Particles not captured by the second fluidic cell may be directed to one or more receptacles and represent particles S with size S_{lower}<S<Sᵤₚₚₑᵣ.

In particular embodiments, the multistage filter system may be used to filter a given sample volume for one or more impurities. As an example and not by way of limitation, each filter stage may remove impurities using DEP and a combination of steps not limited to adsorption, absorption, precipitation, and magnetic separation, where each stage may be independently tuned with various DEP parameters including but not limited to electrode geometry, cartridge surface area, thin film coatings, and/or any other suitable parameter to optimize removal of a given impurity or set of impurities.

In particular embodiments, one or more filters of the multistage dielectrophoretic filter system 210 may be modified for EV purification, wherein each stage of the multistage filter system may sequentially purify the biological sample (e.g., plasma 120) against one or more targeted impurities.

In particular embodiments, digital output for one or more biomarkers (e.g., proteins, lipids, carbohydrates, and/or nucleic acids of EVs 102) may be analyzed by one or more machine-learning algorithms. In particular embodiments, a detection system of multistage dielectrophoretic filter system 210 may analyze particular properties of the one or more biomarkers via one or more machine-learning algorithms. As an example and not by way of limitation, the one or more machine-learning algorithms may include supervised, unsupervised, semi-supervised, deep, and/or reinforcement learning algorithms. In particular embodiments, the deep learning algorithms may include any artificial neural networks (ANNs) that may be utilized to learn deep levels of representations and abstractions from large amounts of data. For example, the deep learning algorithms may include ANNs, such as a multilayer perceptron (MLP), an autoencoder (AE), a convolutional neural network (CNN), a recurrent neural network (RNN), long short term memory (LSTM), a grated recurrent unit (GRU), a restricted Boltzmann Machine (RBM), a deep belief network (DBN), a bidirectional recurrent deep neural network (BRDNN), a generative adversarial network (GAN), deep Q-networks, a neural autoregressive distribution estimation (NADE), an adversarial network (AN), attentional models (AM), deep reinforcement learning, and so forth.

In particular embodiments, digital output from the multistage dielectrophoretic filter system 210 for one or more biomarkers (e.g., proteins, lipids, carbohydrates, and/or nucleic acids of EVs 102) may be analyzed by one or more classification machine-learning algorithms or functions which may include any algorithms that may utilize a supervised learning model (e.g., logistic regression, naïve Bayes, stochastic gradient descent (SGD), k-nearest neighbors, decision trees, random forests, support vector machine (SVM), and so forth) to learn from the data input to the supervised learning model and to make new observations or classifications based thereon.

Although this disclosure references the forementioned machine-learning algorithms, this disclosure contemplates any suitable machine-learning algorithm. In particular embodiments, the one or more machine-learning algorithms may analyze a wide variety of data, allowing for detection of early cancer (e.g., Stage I, Stage II) and/or other diseases of interest.

FIG. 2B illustrates an example diagram 250 of a bottom-view of a multistage dielectrophoretic filter system 210. In particular embodiments, the multistage dielectrophoretic filter system 210 may be programmed to filter EVs 102 between 30 nm and 10,000 nm. In particular embodiments, a multistage dielectrophoretic filter system 210 may include one or more physical housings 212, 214, one or more electronic boards, one or more electrode configurations (e.g., electrode configurations 220, 222), fluidics machinery, one or more tubing channels, one or more reservoirs for reagents, one or more mechanisms for delivering biological sample input and/or accepting biological sample output (e.g., tube 260), reservoirs containing the multistage DEP filter system, and one or more mechanisms for accepting sample inputs and/or flowing sample outputs (e.g., eluates) to one or more containers for storage, shipment, and/or subsequent analysis. In particular embodiments, tube 260 may deliver biological sample input and/or deliver biological sample output. As used here in, electrode configurations 220, 222 may refer to configurations of electrode arrays. Although FIG. 2B discloses a particular electrode configuration 220, 222, this disclosure contemplates that a multistage dielectrophoretic filter system 210 may contain any suitable number or configuration of electrodes and/or electrode arrays.

In particular embodiments, the multistage dielectrophoretic filter system 210 may contain two or more physical housings 212, 214, wherein one or more physical housings 212, 214 may receive input biological samples (e.g., plasma 120), and one or more physical housings 212, 214 may output the biological sample into one or more containers for storage, shipment, and/or subsequent analysis.

In particular embodiments, the purification system may be constructed out of organic thin films or any other suitable material. As an example and not by way of limitation, the organic thin films are polymer films. As another example and not by way of limitation, the polymer films are engineered to have hydrophilicity and/or adsorptive properties that enable gettering (filtering, capturing, or trapping) certain molecules in the sample. In particular embodiments, the thin film may made of inorganic materials. As an example and not by way of limitation, the inorganic materials can be engineered to have pores that can also getter molecules in the sample. In particular embodiments, the thin film may made of a combination or organic and inorganic materials.

In particular embodiments, the multistage DEP filter system 210 can further comprise a computer in operable communication with the electronic control board. In particular embodiments, the computer allows a user to operate the DEP filter system. In particular embodiments, the computer allows automated operation of the DEP filter system. In an example embodiment, the computer may comprise at least one processor; at least one memory; an I/O interface; a display; at least one communication interface; and a library of algorithms tangibly stored in the memory and executable by the processor, said algorithms comprising processor-executable instructions not limited to actuating one or more of the plurality of fluid transfer devices, the input fluid transfer device, and the output fluid transfer device.

### 3. Methods of Purifying EVs

The presently disclosed subject matter also provides a method for purifying an EV of interest. One exemplary method comprises obtaining a biological sample comprising EVs; applying the sample into a first fluidic cell in an array of fluidic cells of a multistage dielectrophoretic (DEP) filter system; tuning the fluidic cell using one or more capture parameters suitable for capturing the EV of interest from the sample, but not other biological particles; applying a wash solution into the fluidic cell to move the uncaptured biological particles to a waste reservoir; stopping the tuning to release the captured EV of interest; applying a elution solution to the fluidic cell; applying the eluted EV of interest to a successive fluidic cell in the array; repeating the steps tuning, wash solution application, stopping the tuning, elution solution application, and applying the eluted EV of interest to a successive fluidic cell, n times, where n is the total number of fluidic cells in the array-1; eluting the EV of interest from the last fluidic cell in the array.

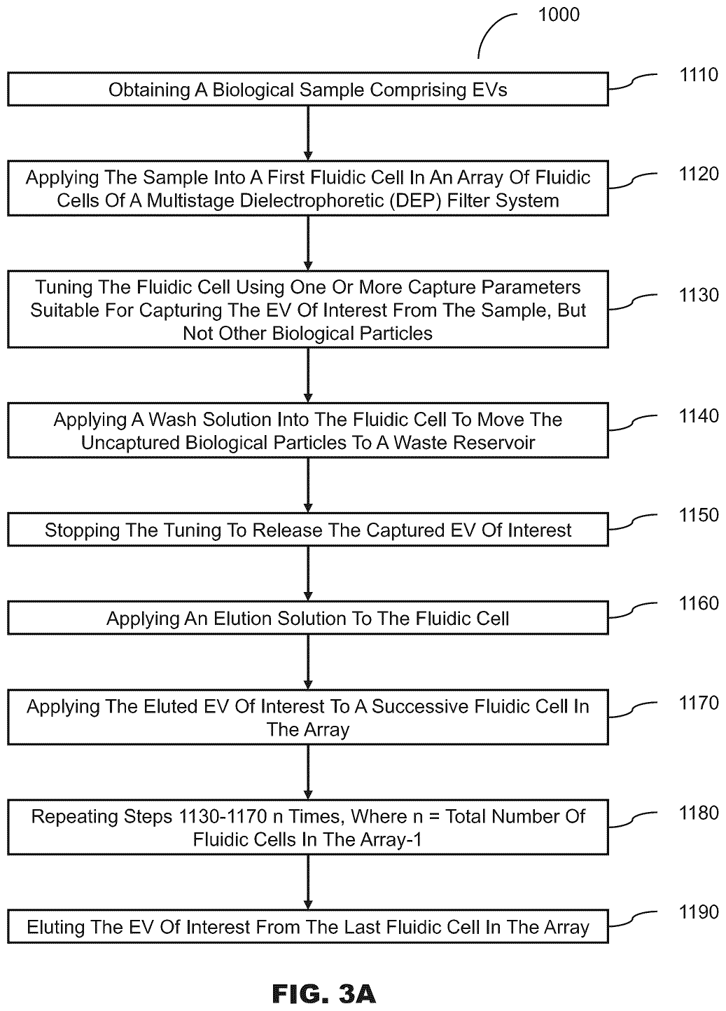

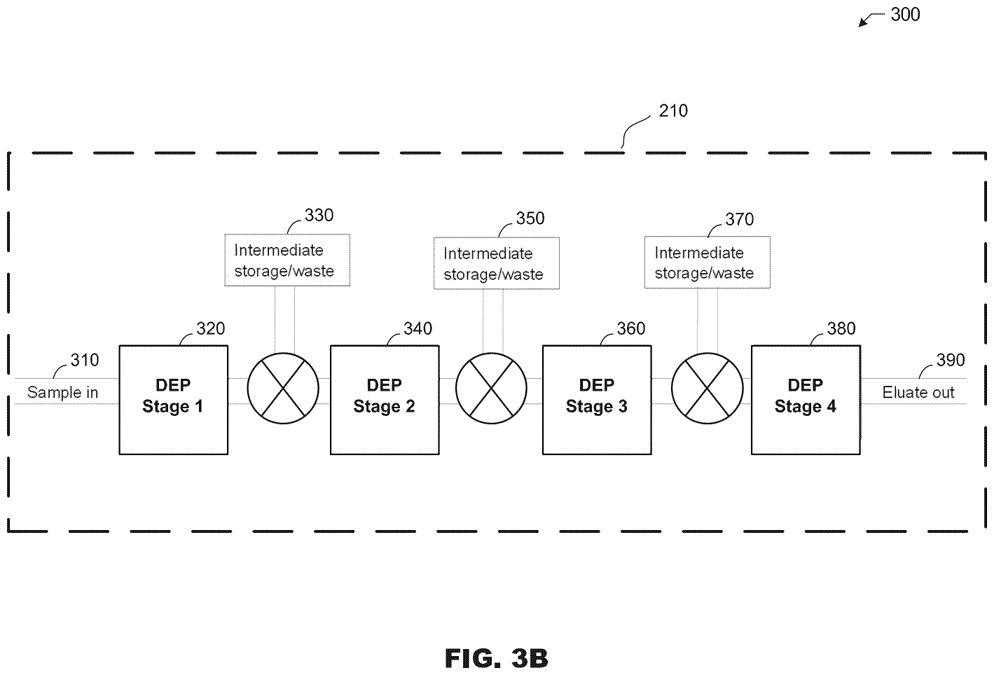

FIG. 3A illustrates an example method 1000 for purifying an EV of interest. The method may begin at step 1110, where the method may comprise obtaining a biological sample comprising EVs. At step 1120, the method may comprise. At step 1130, the method may comprise tuning the fluidic cell using one or more capture parameters suitable for capturing the EV of interest from the sample, but not other biological particles. At step 1140, the method may comprise applying a wash solution into the fluidic cell to move the uncaptured biological particles to a waste reservoir. At step 1150, the method may comprise stopping the tuning to release the captured EV of interest. At step 1160, the method may comprise applying an elution solution to the fluidic cell. At step 1170, the method may comprise applying the eluted EV of interest to a successive fluidic cell in the array. At step 1180, the method may comprise repeating steps 1130-1170 n times, where n=total number of fluidic cells in the array-1. At step 1190, the method may comprise eluting the EV of interest from the last fluidic cell in the array. Particular embodiments may repeat one or more steps of the method of FIG. 3A, where appropriate. Although this disclosure describes and illustrates particular steps of the method of FIG. 3A as occurring in a particular order, this disclosure contemplates any suitable steps of the method of FIG. 3A occurring in any suitable order. Moreover, although this disclosure describes and illustrates an example method for purifying an EV of interest including the particular steps of the method of FIG. 3A, this disclosure contemplates any suitable method for purifying an EV of interest including any suitable steps, which may include all, some, or none of the steps of the method of FIG. 3A, where appropriate. Furthermore, although this disclosure describes and illustrates particular components, devices, or systems carrying out particular steps of the method of FIG. 3A, this disclosure contemplates any suitable combination of any suitable components, devices, or systems carrying out any suitable steps of the method of FIG. 3A.

In particular embodiments, the biological sample is plasma, blood, a liquid biopsy, a cell lysate, or a tissue lysate.

FIG. 3B illustrates an example flowchart 300 of a multistage dielectrophoretic filter system 210. Although FIG. 3B discloses a four (4) stage filter system, this disclosure contemplates a filter system with any suitable number of stages. In particular embodiments, the multistage dielectrophoretic filter process may begin at step 310, wherein one or more biological samples (e.g., plasma 120) may be input to the multistage dielectrophoretic filter system 210 via one or more input ports of the multistage dielectrophoretic filter system 210.

In particular embodiments, at step 320, the one or more biological samples may be input to a first DEP chamber (e.g., DEP Stage 1) via fluidics machinery, medical equipment (e.g., syringe), and/or one or more mechanisms for accepting biological sample inputs. Although this disclosure discusses the aforementioned methods of inputting a biological sample into one or more DEP chambers of the filter system, this disclosure contemplates any suitable method of delivering one or more biological samples into the filter system. In particular embodiments, the transfer of fluids is facilitated using a fluid transfer device selected from a syringe, a pump, and an automated liquid controller. As an example and not by way of limitation, the transfer of fluids is facilitated using a fluid transfer device selected from: a syringe, an actuator in operable communication with the syringe, a valve in operable communication with the actuator and a motor in operable communication with the valve.

In particular embodiments, two or more filters of the multistage dielectrophoretic filter system 210 may be employed in an asymmetric fashion. In particular embodiments, one or more filters of the multistage dielectrophoretic filter system 210 may be employed in a symmetric fashion. For example, two or more DEP filters of the multistage dielectrophoretic filter system 210 may be identical DEP filters linked in series. As an example and not by way of limitation, when two or more symmetric or asymmetric filters are placed in a series, the succession of filters may result in an overall improvement in system efficiency.

In particular embodiments, the multistage dielectrophoretic filter system 210 may be a band-pass filter system, comprising multiple non-identical filter systems (each containing one or more DEP filter stages), wherein each of the filter systems may have different particle capture characteristics. As an example and not by way of limitation, the particle capture characteristics may be determined by a combination of settings for electrode geometry, frequency, voltage, surface coating, medium, and/or particle composition.

In particular embodiments, at step 320, the biological sample (e.g., plasma 120) may be processed by a first DEP chamber (e.g., DEP Stage 1) containing one or more particular electrode configurations and/or arrays (e.g., electrode configuration 220, 222). In particular embodiments, at step 320, one or more sets of operating conditions and/or parameters may be selected for the first DEP chamber. As an example and not by way of limitation, one or more DEP chambers may be tuned via parameters such as frequency, voltage, field source geometry (e.g., electrode and insulator pattern), fluid medium, electrode pitch, solution conductivity, electrode geometry, surface coatings, and/or any other suitable parameter to attract particles of a desired range and repel particles outside of the particular range. In particular embodiments, surface coatings may be chosen and/or modified to set layer thickness, porosity, and/or hydrophilicity.

In particular embodiments, at step 320, the biological sample (e.g., plasma 120) may be flowed over the electrodes of the first DEP chamber, wherein the electrodes of one or more electrode configurations 220, 222 may generate an electric field, resulting in the capture of one or more EVs 102. In particular embodiments, remaining EVs uncaptured by the electric field may be carried away in a wash volume, wherein the wash volume may be directed through one or more additional filters and/or stages, where the wash volume may have additional opportunities to be recaptured and retained.

In particular embodiments, at step 330, one or more fluidics systems of the multistage dielectrophoretic filter system 210 may be engaged to move the biological sample (e.g., plasma 120), one or more reagents, and/or wash volume from step 320 to an intermediate storage and/or waste system.

In particular embodiments, at step 340, the biological sample (e.g., plasma 120) may be input to a second DEP chamber (e.g., DEP Stage 2) of the multistage dielectrophoretic filter system 210. As an example and not by way of limitation, DEP Stage 2 may be comprised of a purification system. In this example, the filter of DEP Stage 2 may remove impurities from the biological sample. In particular embodiments, the second DEP chamber may be tuned to one or more parameters. As an example and not by way of limitation, the second DEP chamber may be tuned via parameters such as frequency, voltage, solution conductivity, electrode geometry, surface coatings, and/or any other suitable parameter to attract particles of a desired range and repel particles outside of the range.

In particular embodiments, at step 350, one or more fluidics systems of the multistage dielectrophoretic filter system 210 may be engaged to move the biological sample (e.g., plasma 120), one or more reagents, and/or the wash volume from step 340 to be stored in an intermediate storage and/or waste system.

In particular embodiments, at step 360, fluid (e.g., biological sample, one or more reagents, and/or wash volume) may be input to a third DEP chamber (third fluidic cell) of the multistage dielectrophoretic filter system 210. As an example and not by way of limitation, the third fluidic cell may be programmed to act as a band-pass filter, wherein the band-pass filtration may be tuned to accept or reject particles of a desired size or within a range of sizes. As an example and not by way of limitation, the band-pass filter may exclude smaller nanoparticles in the third fluidic cell by using predefined DEP parameters and repel large particles in a later stage with different DEP parameters.

In particular embodiments, the third DEP chamber may be tuned to one or more parameters. As an example and not by way of limitation, the third DEP chamber may be tuned via parameters such as frequency, voltage, solution conductivity, electrode geometry, surface coatings, and/or any other suitable parameter to attract particles of a desired range and repel particles outside of the desired range.

In particular embodiments, at step 370, one or more fluidics systems of the multistage dielectrophoretic filter system 210 may be engaged to move the biological sample (e.g., plasma 120), one or more reagents, and the wash volume from step 320 to be processed in an intermediate storage and/or waste.

In particular embodiments, at step 380, the biological sample (e.g., plasma 120) may be input to a fourth DEP chamber (e.g., DEP Stage 4) of the multistage dielectrophoretic filter system 210. In particular embodiments, the fourth DEP chamber may be tuned to one or more particular parameters. In particular embodiments, the fourth DEP chamber may be tuned to one or more particular parameters. As an example and not by way of limitation, the fourth DEP chamber may be tuned via parameters such as frequency, voltage, solution conductivity, electrode geometry, surface coatings, and/or any other suitable parameter to attract particles of a desired range and repel particles outside of the range.

In particular embodiments, at step 390, the biological sample/eluate may be output from DEP Stage 4 after being processed, as in step 380.

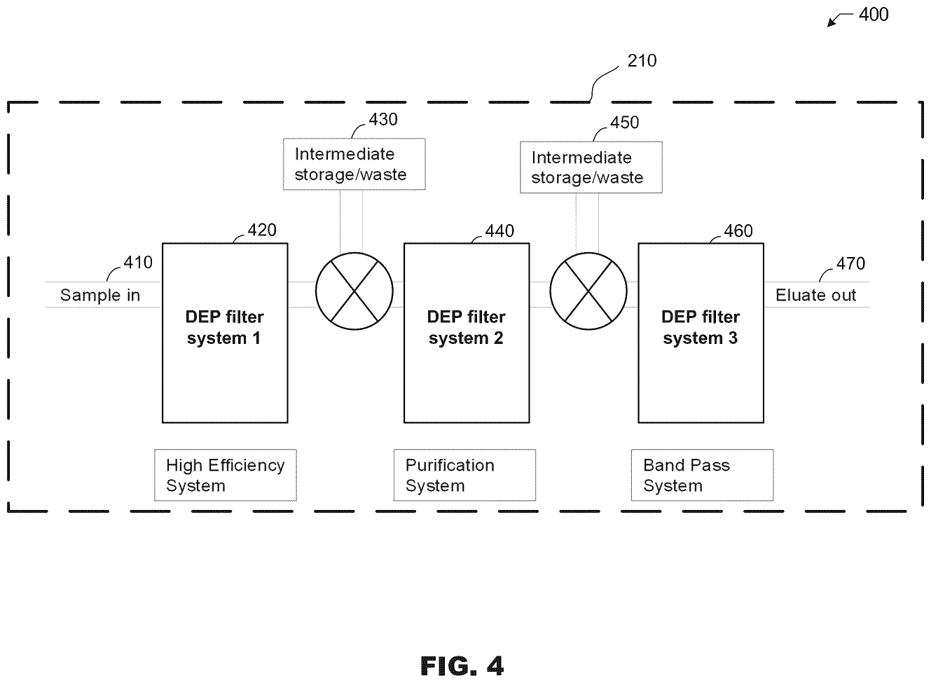

FIG. 4 illustrates an example flowchart 400 of multistage dielectrophoretic filter system 210. Although FIG. 4 discloses a three (3) stage filter system, this disclosure contemplates a filter system with any suitable number of stages. In particular embodiments, the multistage dielectrophoretic filter process may begin at step 410, wherein one or more biological samples (e.g., plasma 120) may be input to the multistage dielectrophoretic filter system 210 via one or more input ports of the multistage dielectrophoretic filter system 210.

In particular embodiments, at step 420, the one or more biological samples (e.g., plasma 120) may be input to a first DEP filter system (e.g., DEP filter system 1) via fluidics machinery, medical equipment (e.g., syringe), and/or one or more mechanisms for accepting biological sample inputs. Although this disclosure discusses the aforementioned methods of inputting a biological sample into one or more DEP chambers of the filter system, this disclosure contemplates any suitable method of delivering one or more biological samples into the filter system. For example, in particular embodiments, the transfer of fluids is facilitated using a fluid transfer device selected from: a syringe, an actuator in operable communication with the syringe, a valve in operable communication with the actuator and a motor in operable communication with the valve.

In particular embodiments, two or more filter systems of the multistage dielectrophoretic filter system 210 may be employed in an asymmetric fashion. In particular embodiments, two or more DEP filter systems of the multistage dielectrophoretic filter system 210 may be employed in a symmetric fashion. For example, two or more DEP filter systems of the multistage dielectrophoretic filter system 210 may be identical DEP filters linked in series. As an example and not by way of limitation, when two or more symmetric or asymmetric filter systems are placed in a series, the succession of filters may result in an overall improvement in system efficiency.

In particular embodiments, the first DEP filter system (first fluidic cell) of step 420, may be a high efficiency system. As an example and not by way of limitation, a high efficiency system can be optimized to produce a very high recovery (>70%) of all extracellular vesicles (EVs) above 50 nm diameter and below 300 nm diameter. In particular embodiments, the first DEP filter system may contain one or more multiple non-identical or identical filters, wherein each of the filters may have different particle capture characteristics. As an example and not by way of limitation, the particle capture characteristics may be determined by a combination of settings directed towards electrode geometry, frequency, voltage, surface coating, medium, and/or particle composition.

In particular embodiments, at step 420, the biological sample may be processed by the first DEP filter system (first fluidic cell), wherein the first DEP filter system may contain one or more particular electrode configurations and/or arrays (e.g., electrode configuration 220, 222). In particular embodiments, at step 420, one or more sets of operating conditions and/or parameters may be selected for the first DEP filter system. As an example and not by way of limitation, one or more DEP filter systems may be tuned via parameters such as frequency, voltage, field source geometry (e.g., electrode and insulator pattern), fluid medium, electrode pitch, solution conductivity, electrode geometry, surface coatings, and/or any other suitable parameter to attract particles of a desired range and repel particles outside of the range. In particular embodiments, surface coatings may be chosen and/or modified to set layer thickness, porosity, and/or hydrophilicity.

In particular embodiments, at step 420, the biological sample (e.g., plasma 120) may be flowed over electrodes of the first DEP filter system (first fluidic cell), wherein the electrodes of wherein the electrodes of one or more electrode configurations 220, 222 may generate an electric field, resulting in the capture of one or more EVs 102. In particular embodiments, remaining EVs uncaptured by the electric field may be carried away in a wash volume, wherein the wash volume may be directed through one or more additional filters, where the wash volume may have additional opportunities to be recaptured and retained.

In particular embodiments, at step 430, one or more fluidics systems of the multistage dielectrophoretic filter system 210 may be engaged to move the biological sample (e.g., plasma 120), one or more reagents, and/or wash volume to be stored in an intermediate storage and/or waste system.

In particular embodiments, at step 440, the fluid (e.g., a mixture of plasma 120, one or more reagents, and/or wash volume) may be input to a second DEP filter system (second fluidic cell) of the multistage dielectrophoretic filter system 210. As an example and not by way of limitation, the second DEP filter system (second fluidic cell) of the multistage dielectrophoretic filter system of step 440 may be a purification system. In this example, the second fluidic cell may remove impurities from the biological sample. In particular embodiments, the purification system may be constructed out of organic thin films or any other suitable material. In particular embodiments, the second DEP filter system may be tuned to one or more parameters. As an example and not by way of limitation, the second DEP filter system may be tuned via parameters such as frequency, voltage, solution conductivity, electrode geometry, surface coatings, and/or any other suitable parameter to attract particles of a desired range and repel particles outside of the range.

In particular embodiments, at step 450, one or more fluidics systems of the multistage dielectrophoretic filter system 210 may be engaged to move the biological sample, one or more reagents, and/or the wash volume from step 440 may be stored in an intermediate storage and/or waste system.

In particular embodiments, at step 460, the fluid (e.g., biological sample, one or more reagents, and/or wash volume) may be input to a third DEP filter system (e.g., DEP filter system 3) of the multistage dielectrophoretic filter system 210. As an example and not by way of limitation, the third DEP filter system may be programmed to act as a band-pass system. As an example and not by way of limitation, the band-pass filter system of step 460, may be tuned to accept and/or reject particles of a desired size or within a desired size range. As an example and not by way of limitation, the band-pass filter system may exclude smaller EVs that pass through the third DEP chamber by using predefined DEP parameters that repel large particles.

In particular embodiments, the third DEP filter system may be tuned to one or more parameters. As an example and not by way of limitation, the third DEP filter system may be tuned via parameters such as frequency, voltage, solution conductivity, electrode geometry, surface coatings, and/or any other suitable parameter to attract particles of a desired range and repel particles outside of the range.

In particular embodiments, at step 470, one or more fluidics systems of the multistage dielectrophoretic filter system 210 may be engaged to move the eluate, as output by step 460, out of the multistage dielectrophoretic filter system 210.

In particular embodiments, the biological sample may be pre-processed/clarified before it is applied to the fluidic cell. As an example and not by way of limitation, the pre-processing may include removal of whole cells, cell debris, and/or aggregates.

### 3.1. Recovery and Purity

In particular embodiments, the DEP filter systems in the disclosed subject matter provide a recovery of EVs in a range from approximately 15% to approximately 99%. In particular embodiments, the DEP filter systems provide a recovery of EVs in a range from approximately 15% to approximately 30%. In particular embodiments, the DEP filter systems provide a recovery of EVs in a range from approximately 20% to approximately 40%. In particular embodiments, the DEP filter systems provide a recovery of EVs in a range from approximately 30% to approximately 50%. In particular embodiments, the DEP filter systems provide a recovery of EVs in a range from approximately 40% to approximately 60%. In particular embodiments, the DEP filter systems provide a recovery of EVs in a range from approximately 50% to approximately 70%. In particular embodiments, the DEP filter systems provide a recovery of EVs in a range from approximately 60% to approximately 80%. In particular embodiments, the DEP filter systems provide a recovery of EVs in a range from approximately 70% to approximately 90%. In particular embodiments, the DEP filter systems provide a recovery of EVs in a range from approximately 80% to approximately 99%. For example, the DEP filter systems described herein can yield a recovery up to approximately 90%, up to approximately 95%, or up to approximately 99%. For example, in particular embodiments the DEP filter systems described herein can provided a progressive increase in recovery as the EVs are progressively enriched from one fluidic cell to the subsequent fluidic cells, to up to approximately 80%, up to approximately 85%, up to approximately 90%, up to approximately 95%, or up to approximately 99%.

In particular embodiments, the DEP filter systems in the disclosed subject matter provide a recovery of EVs that is approximately 2-fold higher to approximately 10-fold higher compared to EVs in the input sample when the DEP filter system is not used. In particular embodiments, the DEP filter systems provide a recovery of EVs that is approximately 2-fold higher to approximately 5-fold higher compared to EVs in the input sample when the DEP filter system is not used. In particular embodiments, the DEP filter systems provide a recovery of EVs that is approximately 4-fold higher to approximately 6-fold higher compared to EVs in the input sample when the DEP filter system is not used. In particular embodiments, the DEP filter systems provide a recovery of EVs that is approximately 5-fold higher to approximately 7-fold higher compared to EVs in the input sample when the DEP filter system is not used. In particular embodiments, the DEP filter systems provide a recovery of EVs that is approximately 6-fold higher to approximately 8-fold higher compared to EVs in the input sample when the DEP filter system is not used. In particular embodiments, the DEP filter systems provide a recovery of EVs that is approximately 7-fold higher to approximately 10-fold higher compared to EVs in the input sample when the DEP filter system is not used. For example, the DEP filter systems described herein provides an approximately 3-fold, approximately 4-fold, or approximately 5-fold recovery of EVs compared to EVs in the input sample when the DEP filter system is not used. For example, in a non-limiting embodiment, the DEP filter systems described herein provides an approximately 4-fold recovery of EVs compared to EVs in the input sample when the DEP filter system is not used.

In particular embodiments, the DEP filter systems in the disclosed subject matter yield an EV of interest in a purity ranging from approximately 15% to approximately 99%. In particular embodiments, the DEP filter systems yield an EV of interest in a purity ranging from approximately 15% to approximately 30%. In particular embodiments, the DEP filter systems yield an EV of interest in a purity ranging from approximately 20% to approximately 40%. In particular embodiments, the DEP filter systems yield an EV of interest in a purity ranging from approximately 30% to approximately 50%. In particular embodiments, the DEP filter systems yield an EV of interest in a purity ranging from approximately 40% to approximately 60%. In particular embodiments, the DEP filter systems yield an EV of interest in a purity ranging from approximately 50% to approximately 70%. In particular embodiments, the DEP filter systems yield an EV of interest in a purity ranging from approximately 60% to approximately 80%. In particular embodiments, the DEP filter systems yield an EV of interest in a purity ranging from approximately 70% to approximately 90%. In particular embodiments, the DEP filter systems yield an EV of interest in a purity ranging from approximately 80% to approximately 99%. For example, the DEP filter systems described herein can yield an EV of interest in a purity up to approximately 90%, up to approximately 95%, or up to approximately 99%. For example, in particular embodiments the DEP filter systems described herein can yield an EV of interest in a purity that increases progressively as the EVs are progressively enriched from one fluidic cell to the subsequent fluidic cells, up to approximately 80%, up to approximately 85%, up to approximately 90%, up to approximately 95%, or up to approximately 99%.

### 4. Extracellular Vesicle (EV) Analysis Systems

The presently disclosed subject matter also provides a system for analyzing the EVs purified/filtered using the DEP filter system and methods described above. One such exemplary analysis system (detection apparatus) comprises, a manifold; a plurality of solenoid actuated values in fluid communication with the manifold; a plurality of analyte reservoirs; one or more analyte isolation and/or tagging chambers; one or more analyte sensor chambers; and one or more fluid transfer devices.

In particular embodiments, the analysis system further comprises a housing that houses the components described above.

In particular non-limiting embodiments, the biomarker expression profile is determined using immunofluorescence, western blotting, hybridization, PCR, mass spectrometry, bioanalyzer, nucleic acid sequencing, silver staining, single particle interferometric reflectance imaging sensor (SP-IRIS), or a combination thereof.

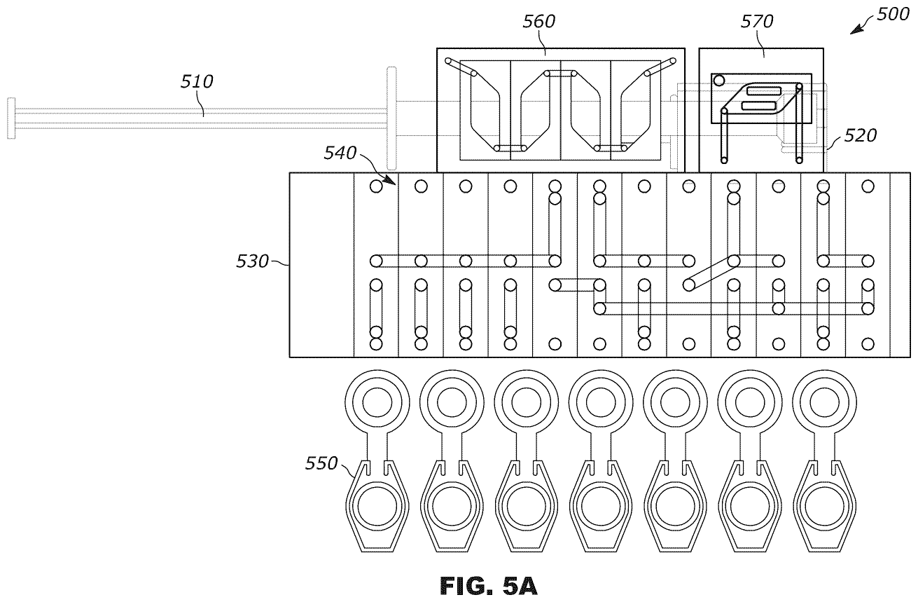

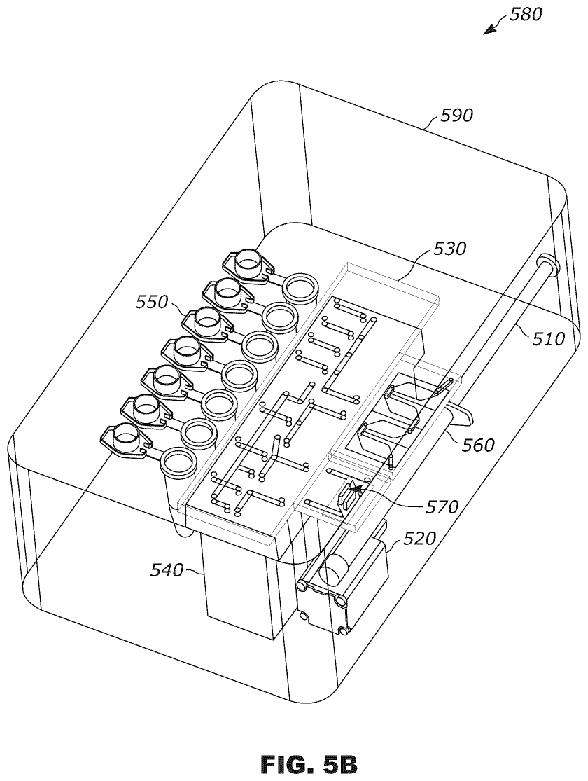

FIG. 5A illustrates an example isometric diagram 500 of a generalized fluidics delivery approach of a system comprising the multistage dielectrophoretic (DEP) filter system 560 (same as 210 in FIGS. 2A and 2B) and a detection apparatus 570, comprising one or more sensors. In particular embodiments, the detection apparatus (e.g., multistage dielectrophoretic filter system 570) may be a consumable chip (e.g., printed circuit board), wherein one or more biological samples (e.g., fluid) may be injected into the detection apparatus by one or more syringes 510.

In particular embodiments, one or more syringes 510 of the detection apparatus may be actuated by one or more motors 520, wherein motor 520 may contain one or more gears. In particular embodiments, the detection apparatus may move fluid (e.g., a biological sample) through manifold 530 to a connected plurality of solenoid actuated values 540. It is understood that the solenoid actuated valves may be arranged in parallel, series, or any other suitable configuration.

In particular embodiments, the detection apparatus may move fluid through one or more reservoirs 550, wherein the one or more reservoirs 550 may be constructed by centrifuge tubes or any other suitable material. In particular embodiments, one or more syringes 510 of the detection apparatus may be actuated by one or more motors 520. In certain embodiments, motor 520 may contain one or more gears. In particular embodiments, the detection apparatus may move fluid (e.g., a biological sample) through manifold 530 to a connected plurality of solenoid actuated valves 540. It is understood that the solenoid actuated valves may be arranged in parallel, series, or any other suitable configuration. In particular embodiments, the detection apparatus may move fluid through one or more reservoirs 550, wherein the one or more reservoirs 550 may be constructed by centrifuge tubes or any other suitable material.

In particular embodiments, the detection apparatus may move fluid through one or more isolation and/or tagging chambers 560, wherein tagging of the fluid and/or isolation of the fluid may occur. As an example and not by way of limitation, the one or more isolation and/or tagging chambers 560 may tag particular chemical groups.

In particular embodiments, the detection apparatus may move fluid through and one or more sensor chambers 570. As an example and not by way of limitation, the detection apparatus may receive one or more biological samples via one or more reservoirs 550, wherein the biological sample may be passed to one or more tagging and/or isolation chambers 560 and subsequently passed to one or more sensor chambers 570. Although this disclosure discusses a particular order of processing biological samples within the detection apparatus, this disclosure contemplates any suitable order of processing biological samples within the detection apparatus.

In particular embodiments, particular bulk reagents may be dedicated to one or more specific chambers 560. As an example and not by way of limitation, chambers 560 may include detector chambers, wherein the detector chambers may receive one or more “detector” reagents. As another example and not by way of limitation, particular detector reagents may be input to one or more particular chambers 560, or a particular detector reagent may be common to all of chambers 560. As an example and not by way of limitation, each chamber of the one or more chambers 560 may label a particular biomarker. For example, one chamber 560 may label one particular biomarker, such as “biomarker 1,” wherein another chamber 560 may label “biomarker 2.” In this example, one or more chambers 560 may cleave specific labels, wherein the labels may be chemically bonded to one or more sensors in the detection apparatus 570, and ultimately digitally quantified. In particular embodiments, the one or more chambers 560 may receive fluid (e.g., biological sample) input and output waste from each particular chamber.

FIG. 5B illustrates an example isometric diagram 580 of a generalized fluidics delivery approach. In particular embodiments, the assembly of the detection apparatus as discussed in diagram 500 of FIG. 5A may be enclosed within box 590. As an example and not by way of limitation, box 590 may house the one or more syringes 510, one or more motors 520, manifold 530, one or more solenoid-actuated valves 540, one or more reservoirs 550, one or more isolation and/or tagging chambers 560, and one or more sensor chambers 570.

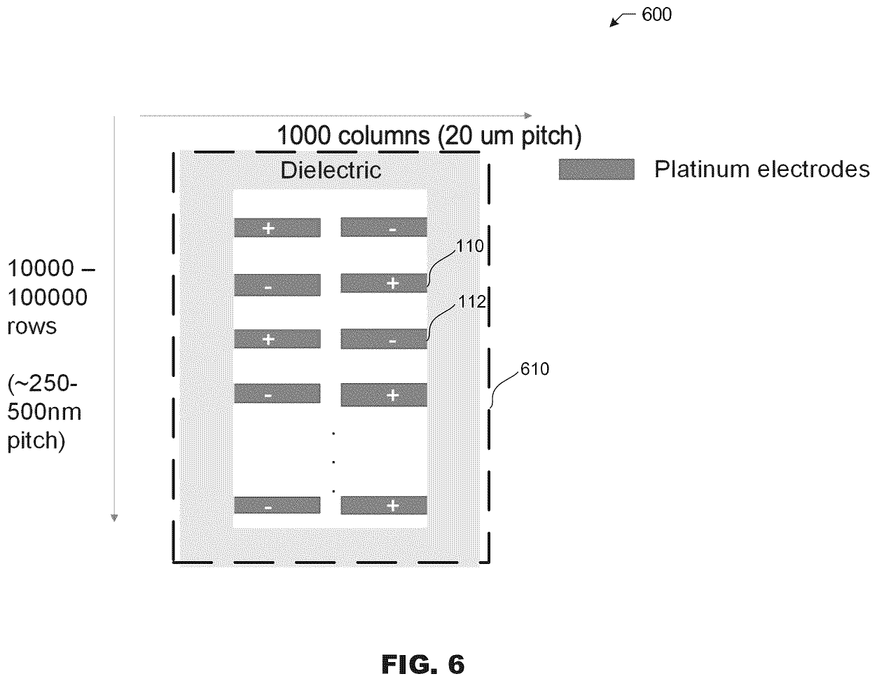

FIG. 6 illustrates an example diagram 600 of a complementary metal-oxide semiconductor array in which one embodiment may operate. In particular embodiments, when an alternating current (AC) waveform is run through positive electrodes 110 and/or negative electrodes 112, the one or more EVs 102 may experience an attractive force that is a function of the RF, voltage, plasma conductivity, EV particle size, and/or EV particle charge. In this example, positive electrodes 110 and/or negative electrodes 112 may be constructed by platinum. Although the disclosure of FIG. 6 discusses positive electrodes 110 and negative electrodes 112 as being constructed by platinum, this disclosure contemplates that positive electrodes 110 and/or negative electrodes 112 may be constructed by a metal or any other suitable material.

In particular embodiments, electrode array 610 may contain a range of 10,000-1,000,000 rows by 1,000 columns of positive electrodes 110 and negative electrodes 112. As an example and not by way of limitation, the rows of electrodes within the electrode array 610 may be constructed at a pitch range of 250-500 nm. As another example and not by way of limitation, the columns of electrodes within the electrode array 610 may be constructed at a pitch of 20 um. Although this disclosure discusses a particular pitch and/or pitch range for both the rows and columns of positive electrodes 110 and negative electrodes 112, this disclosure contemplates any suitable pitch and/or pitch range.

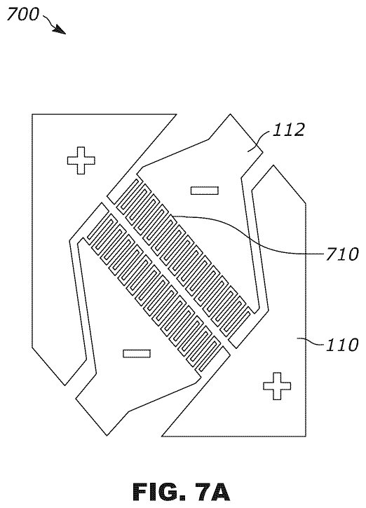

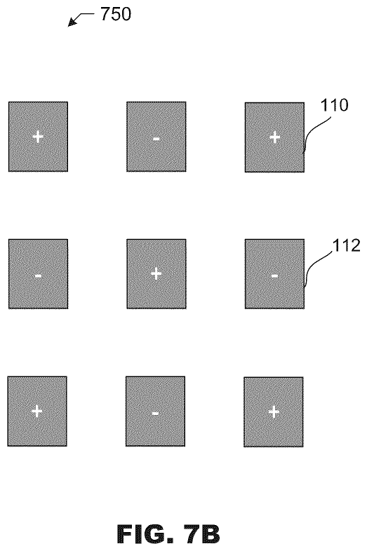

FIG. 7A illustrates an example diagram 700 of an electrode geometry concept. As used herein, interdigitated electrodes (IDEs), referred to hereafter as “interdigitated fingers”, may be defined as one or more electrodes that consists of two sets of closely-spaced fingers separated by gaps on the nm or um scale, and designed to make contact with the surface of a material. In particular embodiments, the biological sample (e.g., plasma 120) may be passed over the electrode array 610 of interdigitated fingers 710, wherein one or more EVs 102 may be captured.

In particular embodiments, the electrode array 610 of interdigitated fingers 710 may be placed on top of dielectric material 114, and comprise an alternating polarity electrode geometry of positive electrodes 110 and negative electrodes 112, wherein the electrodes may be electroformed or sputtered with a pitch between 100 nm and 200 nm. In particular embodiments, the positive electrodes 110 and negative electrodes 112 may operate at a frequency between 100 Hz and 5 MHz and voltage between 0.1 V and 20 V in the biological sample (e.g., plasma, urine, cerebral spinal fluid or any other prepared buffer), with a conductivity between 0.1 mS and 30 mS.

FIG. 7B illustrates an example diagram 750 of an example electrode configuration concept. In particular embodiments, electrode pillars may be arranged in a configuration of columns and/or rows of positive electrodes 110 and negative electrodes 112. In particular embodiments, the electrode pillars may comprise an alternating polarity electrode geometry of positive electrodes 110 and negative electrodes 112, wherein the electrodes may be electroformed or sputtered with a pitch between 100 nm and 200 nm. In particular embodiments, the positive electrodes 110 and negative electrodes 112 may operate at a frequency between 100 Hz and 5 MHZ and voltage between 0.1 V and 20 V in the biological sample (e.g., plasma, urine, cerebral spinal fluid or any other prepared buffer), with a conductivity between 0.1 mS and 30 mS.

### 4.1. Improved Biomarker Quantification

In particular embodiments, the EV analysis systems in the disclosed subject matter provides improved biomarker quantification that is approximately 4 orders of the input sample to approximately 7 orders of the input sample. In particular embodiments, the EV analysis systems provides improved biomarker quantification that is approximately 4 orders of the input sample to approximately 5 orders of the input sample. In particular embodiments, the EV analysis systems provides improved biomarker quantification that is approximately 4.5 orders of the input sample to approximately 5.5 orders of the input sample. In particular embodiments, the EV analysis systems provides improved biomarker quantification that is approximately 5 orders of the input sample to approximately 6 orders of the input sample. In particular embodiments, the EV analysis systems provides improved biomarker quantification that is approximately 5.5 orders of the input sample to approximately 6.5 orders of the input sample. In particular embodiments, the EV analysis systems provides improved biomarker quantification that is approximately 6 orders of the input sample to approximately 7 orders of the input sample.

In particular embodiments, the biomarkers associated with EVs may be membrane bound. In particular embodiments, the biomarkers associated with EVs may located in the lumen of the EVs.

### 5. Integrated Systems for EV Purification and Analysis

The presently disclosed subject matter also provides systems for purifying EVs from a biological sample and analyzing the purified EVs prior to downstream processing. One exemplary system comprises a multistage dielectrophoretic (DEP) filter system; an EV detection/analysis system; and a computer in operable communication with the multistage dielectrophoretic (DEP) filter system and the detection system. In particular embodiments, the multistage dielectrophoretic (DEP) filter system in its various embodiments and associated methods are as described in Sections 2 and 3. In particular embodiments, the EV detection/analysis system in its various embodiments are as described in Section 4.

In particular embodiments, the computer comprises at least one processor; at least one memory; an I/O interface; a display; at least one communication interface; and a library of algorithms tangibly stored in the memory and executable by the processor, said algorithms comprising processor-executable instructions to operate the multistage dielectrophoretic (DEP) filter system and the detection system.

### 5.1. Recovery, Purity, and Improvements in Biomarker Quantification

In particular embodiments, the DEP filter systems in the disclosed subject matter provide a recovery of EVs in a range from approximately 15% to approximately 99%. In particular embodiments, the DEP filter systems provide a recovery of EVs in a range from approximately 15% to approximately 30%. In particular embodiments, the DEP filter systems provide a recovery of EVs in a range from approximately 20% to approximately 40%. In particular embodiments, the DEP filter systems provide a recovery of EVs in a range from approximately 30% to approximately 50%. In particular embodiments, the DEP filter systems provide a recovery of EVs in a range from approximately 40% to approximately 60%. In particular embodiments, the DEP filter systems provide a recovery of EVs in a range from approximately 50% to approximately 70%. In particular embodiments, the DEP filter systems provide a recovery of EVs in a range from approximately 60% to approximately 80%. In particular embodiments, the DEP filter systems provide a recovery of EVs in a range from approximately 70% to approximately 90%. In particular embodiments, the DEP filter systems provide a recovery of EVs in a range from approximately 80% to approximately 99%. For example, the DEP filter systems described herein can yield a recovery up to approximately 90%, up to approximately 95%, or up to approximately 99%. For example, in particular embodiments the DEP filter systems described herein can provided a progressive increase in recovery as the EVs are progressively enriched from one fluidic cell to the subsequent fluidic cells, to up to approximately 80%, up to approximately 85%, up to approximately 90%, up to approximately 95%, or up to approximately 99%.

In particular embodiments, the DEP filter systems in the disclosed subject matter provide a recovery of EVs that is approximately 2-fold higher to approximately 10-fold higher compared to EVs in the input sample when the DEP filter system is not used. In particular embodiments, the DEP filter systems provide a recovery of EVs that is approximately 2-fold higher to approximately 5-fold higher compared to EVs in the input sample when the DEP filter system is not used. In particular embodiments, the DEP filter systems provide a recovery of EVs that is approximately 4-fold higher to approximately 6-fold higher compared to EVs in the input sample when the DEP filter system is not used. In particular embodiments, the DEP filter systems provide a recovery of EVs that is approximately 5-fold higher to approximately 7-fold higher compared to EVs in the input sample when the DEP filter system is not used. In particular embodiments, the DEP filter systems provide a recovery of EVs that is approximately 6-fold higher to approximately 8-fold higher compared to EVs in the input sample when the DEP filter system is not used. In particular embodiments, the DEP filter systems provide a recovery of EVs that is approximately 7-fold higher to approximately 10-fold higher compared to EVs in the input sample when the DEP filter system is not used. For example, the DEP filter systems described herein provides an approximately 3-fold, approximately 4-fold, or approximately 5-fold recovery of EVs compared to EVs in the input sample when the DEP filter system is not used. For example, in a non-limiting embodiment, the DEP filter systems described herein provides an approximately 4-fold recovery of EVs compared to EVs in the input sample when the DEP filter system is not used.

In particular embodiments, the DEP filter systems in the disclosed subject matter yield an EV of interest in a purity ranging from approximately 15% to approximately 99%. In particular embodiments, the DEP filter systems yield an EV of interest in a purity ranging from approximately 15% to approximately 30%. In particular embodiments, the DEP filter systems yield an EV of interest in a purity ranging from approximately 20% to approximately 40%. In particular embodiments, the DEP filter systems yield an EV of interest in a purity ranging from approximately 30% to approximately 50%. In particular embodiments, the DEP filter systems yield an EV of interest in a purity ranging from approximately 40% to approximately 60%. In particular embodiments, the DEP filter systems yield an EV of interest in a purity ranging from approximately 50% to approximately 70%. In particular embodiments, the DEP filter systems yield an EV of interest in a purity ranging from approximately 60% to approximately 80%. In particular embodiments, the DEP filter systems yield an EV of interest in a purity ranging from approximately 70% to approximately 90%. In particular embodiments, the DEP filter systems yield an EV of interest in a purity ranging from approximately 80% to approximately 99%. For example, the DEP filter systems described herein can yield an EV of interest in a purity up to approximately 90%, up to approximately 95%, or up to approximately 99%. For example, in particular embodiments the DEP filter systems described herein can yield an EV of interest in a purity that increases progressively as the EVs are progressively enriched from one fluidic cell to the subsequent fluidic cells, up to approximately 80%, up to approximately 85%, up to approximately 90%, up to approximately 95%, or up to approximately 99%.

In particular embodiments, the EV analysis systems in the disclosed subject matter provides improved biomarker quantification that is approximately 4 orders of the input sample to approximately 7 orders of the input sample. In particular embodiments, the EV analysis systems provides improved biomarker quantification that is approximately 4 orders of the input sample to approximately 5 orders of the input sample. In particular embodiments, the EV analysis systems provides improved biomarker quantification that is approximately 4.5 orders of the input sample to approximately 5.5 orders of the input sample. In particular embodiments, the EV analysis systems provides improved biomarker quantification that is approximately 5 orders of the input sample to approximately 6 orders of the input sample. In particular embodiments, the EV analysis systems provides improved biomarker quantification that is approximately 5.5 orders of the input sample to approximately 6.5 orders of the input sample. In particular embodiments, the EV analysis systems provides improved biomarker quantification that is approximately 6 orders of the input sample to approximately 7 orders of the input sample.

### 6. Methods of Diagnosis

### 6.1. Methods for Assessing Risk or Presence of a Disease or Disorder

The presently disclosed subject matter also provides methods for determining whether a subject is at risk, or suffering from a disease or disorder that needs treatment. One exemplary method for risk assessment comprises obtaining a sample comprising one or more EVs from the subject; purifying one or more EVs from the sample; determining a biomarker expression profile for the purified EVs; and identifying the subject as at risk, or suffering from the disease based on the biomarker expression profile.

In particular non-limiting embodiments, the disease or disorder is selected from a cancer, an autoimmune disease, a vascular disease, a neurodegenerative disease, a metabolic disease, a renal disease, and an inflammatory bowel disease (IBD).

In particular embodiments, purifying one or more EVs from the sample is enabled using the multistage dielectrophoretic (DEP) filter system in its various embodiments, and associated methods as described in Sections 2 and 3.

In particular embodiments, determining a biomarker expression profile for the purified EVs is enabled using the EV detection/analysis system in its various embodiments as described in Section 4. In particular non-limiting embodiments, the biomarker expression profile is determined using immunofluorescence, western blotting, hybridization, PCR, mass spectrometry, bioanalyzer, nucleic acid sequencing, silver staining, single particle interferometric reflectance imaging sensor (SP-IRIS), or a combination thereof.

In particular embodiments, the method further comprises comparing the biomarker expression profile with a reference standard of EV biomarker expression profiles, wherein identifying the subject as at risk, or suffering from the disease is based on the comparison. In a non-limiting example, the reference standard is a database or a chart of EV biomarker expression profiles and their associations with diseases and/or disorders. In a non-limiting example, the reference standard has been established from EV profiling analysis performed in samples obtained from a population of subjects.

In particular embodiments, the method further comprises treating the subject for the disease or disorder.

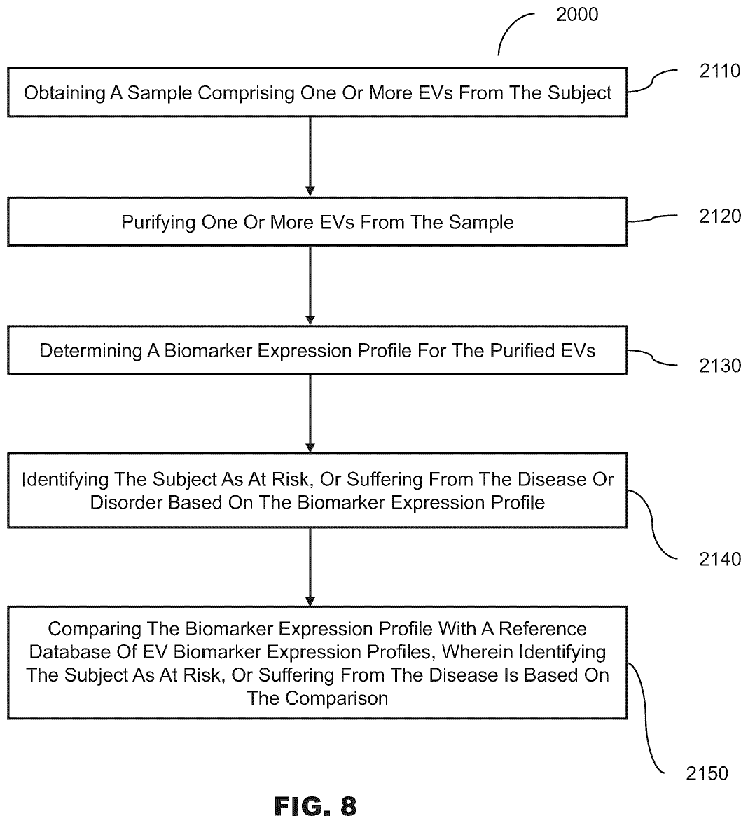

FIG. 8 illustrates an example method 2000 for determining whether a subject is at risk, or suffering from a disease or disorder that needs treatment. The method may begin at step 2110, where the method may comprise obtaining a sample comprising one or more EVs from the subject. At step 2120, the method may comprise purifying one or more EVs from the sample. At step 2130, the method may comprise determining a biomarker expression profile for the purified EVs. At step 2140, the method may comprise identifying the subject as at risk, or suffering from the disease or disorder based on the biomarker expression profile. At step 2150, the method may comprise comparing the biomarker expression profile with a reference database of EV biomarker expression profiles, wherein identifying the subject as at risk, or suffering from the disease is based on the comparison. Particular embodiments may repeat one or more steps of the method of FIG. 8, where appropriate. Although this disclosure describes and illustrates particular steps of the method of FIG. 8 as occurring in a particular order, this disclosure contemplates any suitable steps of the method of FIG. 8 occurring in any suitable order. Moreover, although this disclosure describes and illustrates an example method for determining whether a subject is at risk, or suffering from a disease or disorder that needs treatment including the particular steps of the method of FIG. 8, this disclosure contemplates any suitable method for determining whether a subject is at risk, or suffering from a disease or disorder that needs treatment, including any suitable steps, which may include all, some, or none of the steps of the method of FIG. 8, where appropriate. Furthermore, although this disclosure describes and illustrates particular components, devices, or systems carrying out particular steps of the method of FIG. 8, this disclosure contemplates any suitable combination of any suitable components, devices, or systems carrying out any suitable steps of the method of FIG. 8.

### 6.2. Methods for Monitoring Clinical Status of a Disease or Disorder

The presently disclosed subject matter also provides methods for monitoring the clinical status of a disease or disorder in a subject. One exemplary method comprises obtaining a control sample comprising one or more EVs from the subject; purifying from the control sample, EVs associated with the disease; quantifying a control expression level of one or more biomarkers associated with the disease; obtaining a test sample comprising one or more EVs from the subject; purifying from the test sample, EVs associated with the disease; quantifying an updated expression level of the one or more biomarkers associated with the disease, wherein, a change in updated expression level of the one or more biomarkers compared to the control expression level indicates a change in the clinical status of the disease.

In particular embodiments, the subject has been treated for the disease or disorder before the second obtaining step.

In particular embodiments, a lower updated expression level of the one or more biomarkers compared to the control expression level indicates that the subject is in remission and/or responding to treatment. In particular embodiments, a lower updated expression level of the one or more biomarkers compared to the control expression level indicates that the subject is in relapse and/or not responding to treatment. In particular embodiments, a higher updated expression level of the one or more biomarkers compared to the control expression level indicates that the subject is in remission and/or responding to treatment. In particular embodiments, a higher updated expression level of the one or more biomarkers compared to the control expression level indicates that the subject is in relapse and/or not responding to treatment.

In particular non-limiting embodiments, the disease or disorder is selected from a cancer, an autoimmune disease, a vascular disease, a neurodegenerative disease, a metabolic disease, a renal disease, and an inflammatory bowel disease (IBD).

In particular embodiments, purifying one or more EVs from the sample is enabled using the multistage dielectrophoretic (DEP) filter system in its various embodiments, and associated methods as described in Sections 2 and 3.

In particular embodiments, determining a biomarker expression profile for the purified EVs is enabled using the EV detection/analysis system in its various embodiments as described in Section 4. In particular non-limiting embodiments, the biomarker expression profile is determined using immunofluorescence, western blotting, hybridization, PCR, mass spectrometry, bioanalyzer, nucleic acid sequencing, silver staining, single particle interferometric reflectance imaging sensor (SP-IRIS), or a combination thereof.

In particular embodiments, the one or more biomarkers quantified is selected from a reference database or a reference chart of EV biomarker expression profiles and their associated diseases and/or disorders. In a non-limiting example, the reference standard has been established from EV profiling analysis performed in samples obtained from a population of subjects.

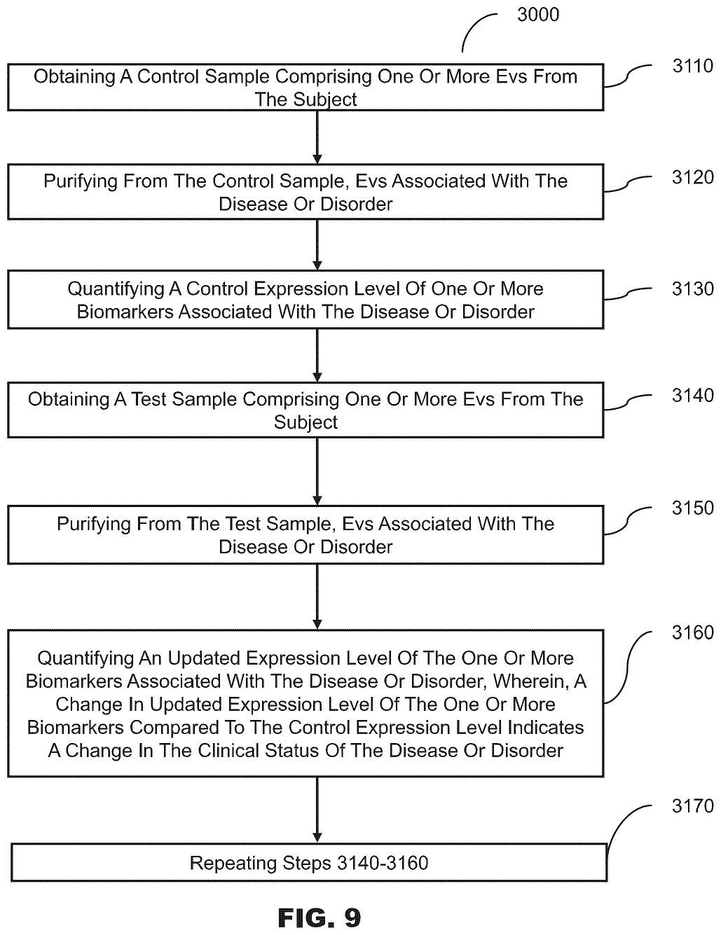

FIG. 9 illustrates an example method 3000 for monitoring the clinical status of a disease or disorder in a subject. The method may begin at step 3110, where the method may comprise obtaining a control sample comprising one or more EVs from the subject. At step 3120, the method may comprise purifying from the control sample, EVs associated with the disease or disorder. At step 3130, the method may comprise quantifying a control expression level of one or more biomarkers associated with the disease or disorder. At step 3140, the method may comprise obtaining a test sample comprising one or more EVs from the subject. At step 3150, the method may comprise purifying from the test sample, EVs associated with the disease or disorder. At step 3160, the method may comprise quantifying an updated expression level of the one or more biomarkers associated with the disease or disorder, wherein, a change in updated expression level of the one or more biomarkers compared to the control expression level indicates a change in the clinical status of the disease or disorder. At step 3170, the method may comprise repeating steps 3140-3160. Particular embodiments may repeat one or more steps of the method of FIG. 9, where appropriate. Although this disclosure describes and illustrates particular steps of the method of FIG. 9 as occurring in a particular order, this disclosure contemplates any suitable steps of the method of FIG. 9 occurring in any suitable order. Moreover, although this disclosure describes and illustrates an example method for monitoring the clinical status of a disease or disorder in a subject including the particular steps of the method of FIG. 9, this disclosure contemplates any suitable method for monitoring the clinical status of a disease or disorder in a subject including any suitable steps, which may include all, some, or none of the steps of the method of FIG. $, where appropriate. Furthermore, although this disclosure describes and illustrates particular components, devices, or systems carrying out particular steps of the method of FIG. 9, this disclosure contemplates any suitable combination of any suitable components, devices, or systems carrying out any suitable steps of the method of FIG. 9.

### 7. Systems and Methods

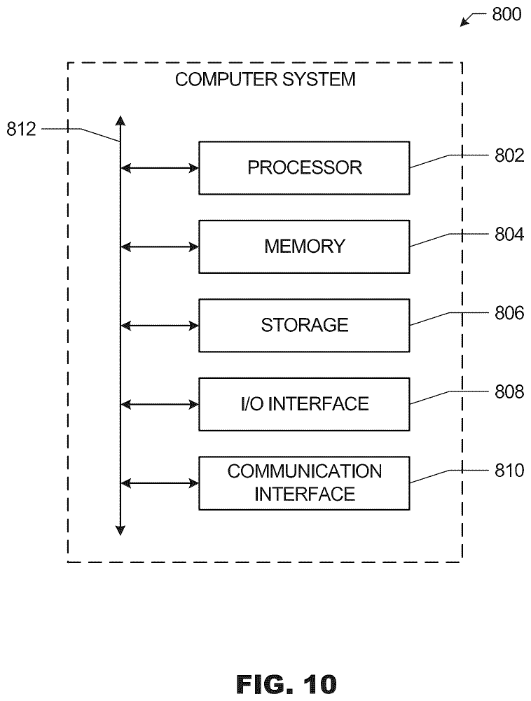

FIG. 10 illustrates an example computer system 800 that may be utilized to perform digital, multiplexed, extracellular vesicle-derived biomarker lab-on-a-chip diagnostics, in accordance with the presently disclosed embodiments. In particular embodiments, one or more computer systems 800 perform one or more steps of one or more methods described or illustrated herein. In particular embodiments, one or more computer systems 800 provide functionality described or illustrated herein. In particular embodiments, software running on one or more computer systems 800 performs one or more steps of one or more methods described or illustrated herein or provides functionality described or illustrated herein. Particular embodiments include one or more portions of one or more computer systems 800. Herein, reference to a computer system may encompass a computing device, and vice versa, where appropriate. Moreover, reference to a computer system may encompass one or more computer systems, where appropriate.

This disclosure contemplates any suitable number of computer systems 800. This disclosure contemplates computer system 800 taking any suitable physical form. As example and not by way of limitation, computer system 800 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, computer system 800 may include one or more computer systems 800; be unitary or distributed; span multiple locations; span multiple machines; span multiple data centers; or reside in a cloud, which may include one or more cloud components in one or more networks.

Where appropriate, one or more computer systems 800 may perform without substantial spatial or temporal limitation one or more steps of one or more methods described or illustrated herein. As an example and not by way of limitation, one or more computer systems 800 may perform in real time or in batch mode one or more steps of one or more methods described or illustrated herein. One or more computer systems 800 may perform at different times or at different locations one or more steps of one or more methods described or illustrated herein, where appropriate.

In particular embodiments, computer system 800 includes a processor 802, memory 804, storage 806, an input/output (I/O) interface 808, a communication interface 810, and a bus 812. Although this disclosure describes and illustrates a particular computer system having a particular number of particular components in a particular arrangement, this disclosure contemplates any suitable computer system having any suitable number of any suitable components in any suitable arrangement. In particular embodiments, processor 802 includes hardware for executing instructions, such as those making up a computer program. As an example and not by way of limitation, to execute instructions, processor 802 may retrieve (or fetch) the instructions from an internal register, an internal cache, memory 804, or storage 806; decode and execute them; and then write one or more results to an internal register, an internal cache, memory 804, or storage 806. In particular embodiments, processor 802 may include one or more internal caches for data, instructions, or addresses. This disclosure contemplates processor 802 including any suitable number of any suitable internal caches, where appropriate. As an example and not by way of limitation, processor 802 may include one or more instruction caches, one or more data caches, and one or more translation lookaside buffers (TLBs). Instructions in the instruction caches may be copies of instructions in memory 804 or storage 806, and the instruction caches may speed up retrieval of those instructions by processor 802.

Data in the data caches may be copies of data in memory 804 or storage 806 for instructions executing at processor 802 to operate on; the results of previous instructions executed at processor 802 for access by subsequent instructions executing at processor 802 or for writing to memory 804 or storage 806; or other suitable data. The data caches may speed up read or write operations by processor 802. The TLBs may speed up virtual-address translation for processor 802. In particular embodiments, processor 802 may include one or more internal registers for data, instructions, or addresses. This disclosure contemplates processor 802 including any suitable number of any suitable internal registers, where appropriate. Where appropriate, processor 802 may include one or more arithmetic logic units (ALUs); be a multi-core processor; or include one or more processors 802. Although this disclosure describes and illustrates a particular processor, this disclosure contemplates any suitable processor.

In particular embodiments, memory 804 includes main memory for storing instructions for processor 802 to execute or data for processor 802 to operate on. As an example and not by way of limitation, computer system 800 may load instructions from storage 806 or another source (such as, for example, another computer system 800) to memory 804. Processor 802 may then load the instructions from memory 804 to an internal register or internal cache. To execute the instructions, processor 802 may retrieve the instructions from the internal register or internal cache and decode them. During or after execution of the instructions, processor 802 may write one or more results (which may be intermediate or final results) to the internal register or internal cache. Processor 802 may then write one or more of those results to memory 804. In particular embodiments, processor 802 executes only instructions in one or more internal registers or internal caches or in memory 804 (as opposed to storage 806 or elsewhere) and operates only on data in one or more internal registers or internal caches or in memory 804 (as opposed to storage 806 or elsewhere).

One or more memory buses (which may each include an address bus and a data bus) may couple processor 802 to memory 804. Bus 812 may include one or more memory buses, as described below. In particular embodiments, one or more memory management units (MMUs) reside between processor 802 and memory 804 and facilitate accesses to memory 804 requested by processor 802. In particular embodiments, memory 804 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 804 may include one or more memory devices 810, where appropriate. Although this disclosure describes and illustrates particular memory, this disclosure contemplates any suitable memory.

In particular embodiments, storage 806 includes mass storage for data or instructions. As an example and not by way of limitation, storage 806 may include a hard disk drive (HDD), a floppy disk drive, flash memory, an optical disc, a magneto-optical disc, magnetic tape, or a Universal Serial Bus (USB) drive or a combination of two or more of these. Storage 806 may include removable or non-removable (or fixed) media, where appropriate. Storage 806 may be internal or external to computer system 800, where appropriate. In particular embodiments, storage 806 is non-volatile, solid-state memory. In particular embodiments, storage 806 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 806 taking any suitable physical form. Storage 806 may include one or more storage control units facilitating communication between processor 802 and storage 806, where appropriate. Where appropriate, storage 806 may include one or more storages 806. Although this disclosure describes and illustrates particular storage, this disclosure contemplates any suitable storage.

In particular embodiments, I/O interface 808 includes hardware, software, or both, providing one or more interfaces for communication between computer system 800 and one or more I/O devices. Computer system 800 may include one or more of these I/O devices, where appropriate. One or more of these I/O devices may enable communication between a person and computer system 800. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 806 for them. Where appropriate, I/O interface 808 may include one or more device or software drivers enabling processor 802 to drive one or more of these I/O devices. I/O interface 808 may include one or more I/O interfaces 808, where appropriate. Although this disclosure describes and illustrates a particular I/O interface, this disclosure contemplates any suitable I/O interface.

In particular embodiments, communication interface 810 includes hardware, software, or both providing one or more interfaces for communication (such as, for example, packet-based communication) between computer system 800 and one or more other computer systems 800 or one or more networks. As an example and not by way of limitation, communication interface 810 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 810 for it.

As an example and not by way of limitation, computer system 800 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example computer system 800 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 800 may include any suitable communication interface 810 for any of these networks, where appropriate. Communication interface 810 may include one or more communication interfaces 810, where appropriate. Although this disclosure describes and illustrates a particular communication interface, this disclosure contemplates any suitable communication interface.

In particular embodiments, bus 812 includes hardware, software, or both coupling components of computer system 800 to each other. As an example and not by way of limitation, bus 812 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 812 may include one or more buses 812, where appropriate. Although this disclosure describes and illustrates a particular bus, this disclosure contemplates any suitable bus or interconnect.

### 8. EXAMPLES

### Example 1. DEP Filtration Systems Improve EV Recovery from Samples

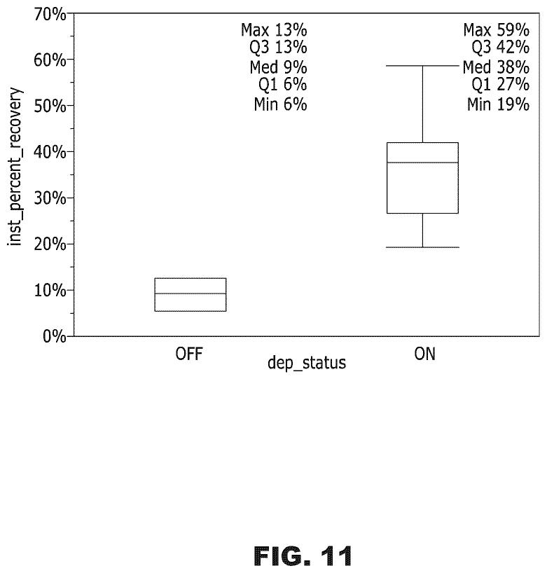

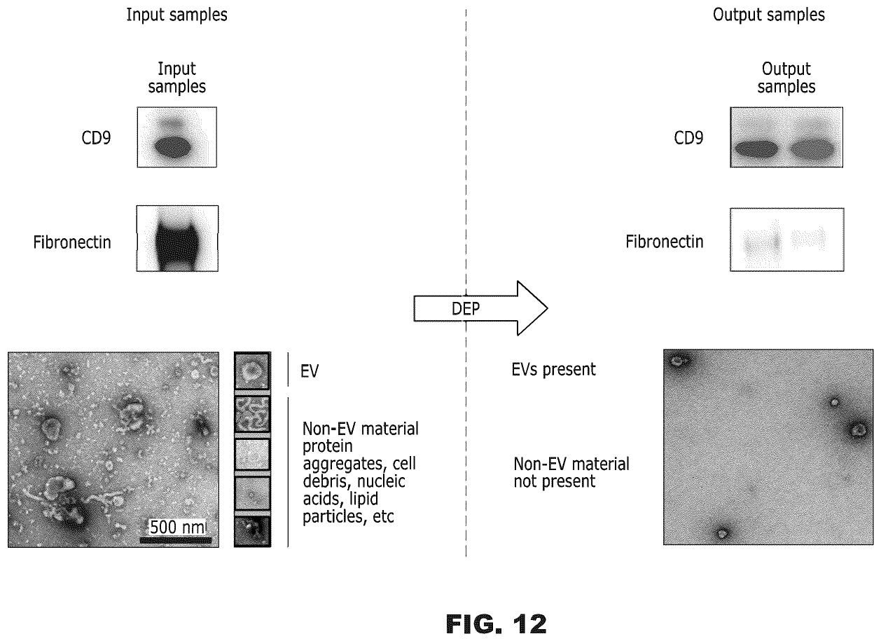

To determine the efficiency of the disclosed DEP filtration systems in improving EV recovery, a conditioned media sample comprising EVs of interest expressing biomarker CD9 and undesired impurities (input sample, control sample) was passed through the DEP filtration system to obtain a test sample (output sample) that was enriched for the EV of interest. EV recovery in the control and test samples (fraction of recovered biomarker sample compared to biomarker signal in the input sample) was quantitated based on the biomarker CD9 signal. As shown in FIG. 11, a 4.2-fold increase in EV recovery was observed for the test sample (Median Recovery, 38%) over the control sample (Median Recovery, 9%). The two states, “OFF” and “ON” in FIG. 11 denote the recovery of the DEP filter system while off (no purification) versus on (post purification). A visual, qualitative analysis of biomarker purity and enrichment revealed a significant enrichment of CD9 and a significant reduction in fibronectin impurity in the test sample compared to the control sample (FIG. 12). The test sample also showed a significant reduction in cell debris, nucleic acids and lipid particle contaminants. Taken together, these data demonstrate the benefits of the disclosed DEP filter system in delivering DEP-specific EV capture.

### Example 2. DEP Filtration Systems Provide Improved Biomarker Quantification

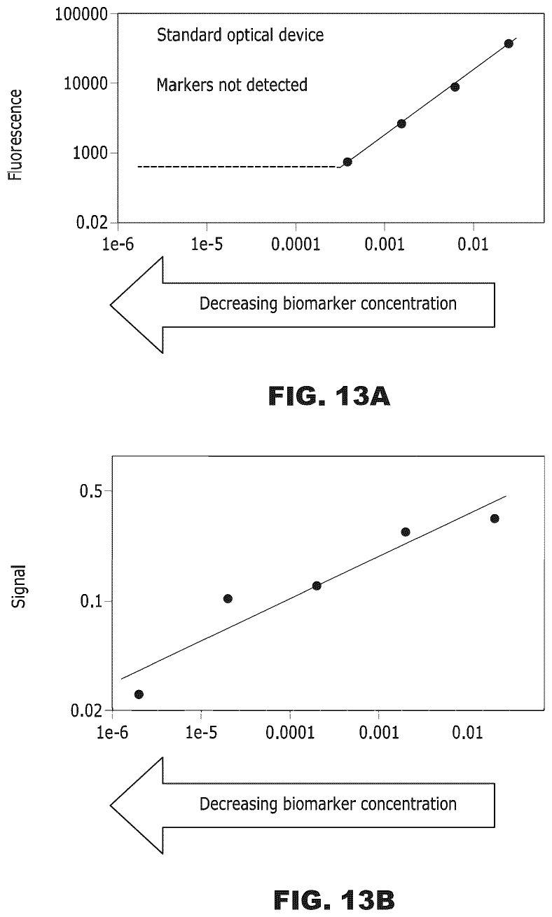

EVs purified using the DEP filtration system disclosed herein were analyzed using industry standard quantification methods and using the EV analysis and quantification system and methods disclosed herein. As shown in FIGS. 13A-13B, while currently used fluorescence-based quantification methods were limited to a biomarker quantification of only 2 orders of input sample concentration, the EV analysis and quantification system and methods yielded a biomarker quantification of over 4.5 orders of input sample concentration that can be extended to at least 7 orders of input sample concentration.

### Example 3. Diagnosis And Monitoring of Diseases From EV-Associated Biomarkers

Sensitive diagnosis of diseases and disorders through biomarker assays sometimes require invasive methods for obtaining a patient sample. While an alternative to such invasive methods would be biomarker assays using a blood sample, currently such assays are insensitive for use in a clinical setting. On the other hand, the ability of the DEP system to separate, purify, and concentrate EVs in a streamlined manner provides a significant advantage over current approaches. For example, extracellular vesicles are able to cross the blood-brain barrier, and therefore can be isolated from a peripheral blood sample. Biomarkers, for example, alpha synuclein (a Parkinson disease biomarker) may be assayed in EVs isolated and purified from peripheral blood using the DEP system and methods disclosed herein. A paired comparison of the existing standard of care cerebrospinal fluid test with EV-associated alpha synuclein between samples from test subjects and healthy individuals can form the basis of utilizing the DEP system for diagnosis in Parkinson's disease. Similarly, EV-associated biomarkers can form the basis of disease monitoring, which would comprise periodic testing of biomarker levels that could establish a change or lack thereof in particular biomarker expression. This might be to monitor the response to a treatment or to chronicle the advancement or remission of a disease. Such strategies may also be used for diagnosis and monitoring of other diseases through screening of EVs for disease specific biomarkers.

### 9. Recitation of Embodiments

Embodiment 1. A multistage dielectrophoretic (DEP) filter system for extracellular vesicles (EVs) purification, comprising: a plurality of housings, each housing comprising: an array of fluidic cells, each comprising an input fluid channel and an output fluid channel, configured to receive and process a sample comprising one or more EVs for purification, each fluidic cell comprising: a semiconductor array comprising a plurality of electrodes; a plurality of fluid transfer devices in fluid communication between two of the array of fluidic cells via the input fluid channel and the output fluid channels, wherein the fluid transfer devices transfer a processed sample between two fluidic cells via the input fluid channel and the output fluid channel; an input fluid transfer device in fluid communication with a first fluidic cell in the array, and an output fluid transfer device in fluid communication with an output fluid channel in a last fluidic cell in the array; one or more fluid actuators; one or more storage containers; at least one electronic control board in operable communication with the plurality of fluid transfer devices, the input fluid transfer device, and the output fluid transfer device; and a power supply in operable communication with the electronic control board.

Embodiment 2. The multistage DEP filter system of Embodiment 1, further comprising a computer in operable communication with the electronic control board, said computer comprising: at least one processor; at least one memory; an I/O interface; a display; at least one communication interface; and a library of algorithms tangibly stored in the memory and executable by the processor, said algorithms comprising processor-executable instructions to actuate one or more of the plurality of fluid transfer devices, the input fluid transfer device, and the output fluid transfer device.

Embodiment 3. The multistage DEP filter system of either of Embodiments 1 or 2, wherein the semiconductor array in the fluidic cell enables operation of the multistage DEP filter system at an operating mode for EV purification that is selected from one or more of, high capture efficiency, purification, or band-pass.

Embodiment 4. The multistage DEP filter system of any one of Embodiments 1-3, wherein the band-pass is based on size of the EV.

Embodiment 5. The multistage DEP filter system of any one of Embodiments 1-4, wherein the electrodes comprise positive electrodes and negative electrodes.

Embodiment 6. The multistage DEP filter system of any one of Embodiments 1-5, wherein the semiconductor array comprises electrodes disposed in one or more electrode configurations, wherein the configuration enable generation of an electric field for capturing the EVs in the sample.

Embodiment 7. The multistage DEP filter system of any one of Embodiments 1-6, wherein one or more of the plurality of fluidic cells have an identical electrode configuration (symmetric electrode configuration).

Embodiment 8. The multistage DEP filter system of any one of Embodiments 1-6, wherein one or more of the plurality of fluidic cells have a non-identical electrode configuration (asymmetric electrode configuration).

Embodiment 9. The multistage DEP filter system of any one of Embodiments 1-6, wherein the electrodes are disposed in an interdigitated electrode (IDE) geometrical pattern.

Embodiment 10. The multistage DEP filter system of any one of Embodiments 1-9, wherein the system is constructed out of organic thin films.

Embodiment 11. The multistage DEP filter system of any one of Embodiments 1-10, wherein one or more of the fluidic cell is coated with an anti-biofouling agent.

Embodiment 12. The multistage DEP filter system of any one of Embodiments 1-11, wherein the fluidics cell is tunable for one or more EV capture parameters.

Embodiment 13. The multistage DEP filter system of any one of Embodiments 1-12, wherein the capture parameters are selected from the group consisting of voltage, frequency, field source geometry, fluid medium, solution conductivity, surface coating, electrode pitch and electrode geometry, or a combination thereof.

Embodiment 14. The multistage DEP filter system of any one of Embodiments 1-13, wherein the fluid transfer device comprises: a syringe; an actuator in operable communication with the syringe; a valve in operable communication with the actuator; and a motor in operable communication with the valve.

Embodiment 15. The multistage DEP filter system of any one of Embodiments 1-14, wherein the valve is a solenoid valve.

Embodiment 16. An extracellular vesicle (EV) analysis system, comprising: a manifold; a plurality of solenoid actuated valves in fluid communication with the manifold; a plurality of analyte reservoirs; one or more analyte isolation and/or tagging chambers; one or more analyte sensor chambers; and one or more fluid transfer devices.

Embodiment 17. The analysis system of Embodiment 16, further comprising a housing that houses the multistage DEP filter system.

Embodiment 18. The analysis system of either of Embodiment 16 or 17, wherein the fluid transfer device is a syringe.

Embodiment 19. The analysis system of any one of Embodiments 16-18, wherein the fluid transfer device comprises: a syringe; an actuator in operable communication with the syringe; a valve in operable communication with the actuator; and a motor in operable communication with the valve.

Embodiment 20. The analysis system of any one of Embodiments 16-19, wherein the valve is a solenoid valve.

Embodiment 21. A system comprising: the multistage dielectrophoretic (DEP) filter system of Embodiment 1; the EV analysis system of Embodiment 16; and a computer in operable communication with the multistage dielectrophoretic (DEP) filter system and the detection system, said computer comprising: at least one processor; at least one memory; an I/O interface; a display; at least one communication interface; and a library of algorithms tangibly stored in the memory and executable by the processor, said algorithms comprising processor-executable instructions to operate the multistage dielectrophoretic (DEP) filter system and the detection system.

Embodiment 22. A method for purifying an EV of interest comprising: (a) obtaining a biological sample comprising EVs; (b) applying the sample into a first fluidic cell in an array of fluidic cells of a multistage dielectrophoretic (DEP) filter system; (c) tuning the fluidic cell using one or more capture parameters suitable for capturing the EV of interest from the sample, but not other biological particles; (d) applying a wash solution into the fluidic cell to move the uncaptured biological particles to a waste reservoir; (e) stopping the tuning to release the captured EV of interest; (f) applying an elution solution to the fluidic cell to elute the EV; (g) applying the eluted EV of interest to a successive fluidic cell in the array; (h) repeating (c)-(g) n times, where n=total number of fluidic cells in the array-1; and (i) eluting the EV of interest from the last fluidic cell in the array.

Embodiment 23. The method of Embodiment 22, further comprising analyzing the eluted EV.

Embodiment 24. The method of either of Embodiments 22 or 23, wherein the analyzing is qualitative and/or quantitative.

Embodiment 25. The method of either of Embodiment 22 or 23, wherein the eluted EV is analyzed using immunofluorescence, western blotting, hybridization, PCR, mass spectrometry, bioanalyzer, nucleic acid sequencing, silver staining, single particle interferometric reflectance imaging sensor (SP-IRIS), or a combination thereof.

Embodiment 26. The method of either of Embodiment 22 or 23, wherein the eluted EV is analyzed using an extracellular vesicle (EV) analysis system.

Embodiment 27. The method of either of Embodiment 22 or 23, wherein the eluted EV is analyzed using the extracellular vesicle (EV) analysis system of Embodiment 16.

Embodiment 28. The method of any one of Embodiments 22-27, further comprising concentrating the eluted EV.

Embodiment 29. The method of any one of Embodiments 22-28, further comprising storing the eluted EV.

Embodiment 30. The method of any one of Embodiments 22-29, wherein the EV is purified using the multistage dielectrophoretic (DEP) filter system of Embodiment 1.

Embodiment 31. The method of any one of Embodiments 22-30, wherein the applying steps are performed using a fluid transfer device selected from a syringe, a pump, and an automated liquid controller.

Embodiment 32. The method of Embodiment 31, wherein the fluid transfer device is a syringe.

Embodiment 33. The method of Embodiment 31, wherein the fluid transfer device comprises: a syringe; an actuator in operable communication with the syringe; a valve in operable communication with the actuator; and a motor in operable communication with the valve.

Embodiment 34. The method of Embodiment 33, wherein the valve is a solenoid valve.

Embodiment 35. The method of any one of Embodiment 22-34, wherein the capture parameters are selected from the group consisting of voltage, frequency, field source geometry, fluid medium, solution conductivity, surface coating, electrode pitch and electrode geometry, and a combination thereof.

Embodiment 36. The method of any one of Embodiment 22-35, wherein the biological sample is plasma, blood, a liquid biopsy, a cell lysate, or a tissue lysate.

Embodiment 37. A method of determining whether a subject is at risk, or suffering from a disease or disorder that needs treatment comprising: obtaining a sample comprising one or more EVs from the subject; purifying one or more EVs from the sample; determining a biomarker expression profile for the purified EVs; and identifying the subject as at risk, or suffering from the disease or disorder based on the biomarker expression profile.

Embodiment 38. The method of Embodiment 37, further comprising comparing the biomarker expression profile with a reference database of EV biomarker expression profiles, wherein identifying the subject as at risk, or suffering from the disease is based on the comparing.

Embodiment 39. The method of either of Embodiment 37 or 38, further comprising treating the subject.

Embodiment 40. The method of any one of Embodiments 37-39, wherein the disease or disorder is selected from the group consisting of a cancer, an autoimmune disease, a vascular disease a neurodegenerative disease, a metabolic disease, a renal disease, and an inflammatory bowel disease (IBD).

Embodiment 41. The method of any one of Embodiments 37-40, wherein the EVs are purified using the system of Embodiment 21.

Embodiment 42. The method of any one of Embodiments 37-41, wherein the EVs are purified using the method of Embodiment 22.

Embodiment 43. The method of any one of Embodiments 37-42, wherein the biomarker expression profile is determined using immunofluorescence, western blotting, hybridization, PCR, mass spectrometry, bioanalyzer, nucleic acid sequencing, silver staining, single particle interferometric reflectance imaging sensor (SP-IRIS), or a combination thereof.

Embodiment 44. A method for monitoring a clinical status of a disease or disorder in a subject comprising: (a) obtaining a control sample comprising one or more EVs from the subject; (b) purifying from the control sample, EVs associated with the disease or disorder; (c) quantifying a control expression level of one or more biomarkers associated with the disease or disorder; (d) obtaining a test sample comprising one or more EVs from the subject; (e) purifying from the test sample, EVs associated with the disease or disorder; and (f) quantifying an updated expression level of the one or more biomarkers associated with the disease or disorder, wherein, a change in updated expression level of the one or more biomarkers compared to the control expression level indicates a change in the clinical status of the disease or disorder.

Embodiment 45. The method of Embodiment 44, further comprising repeating steps (d)-(f).

Embodiment 46. The method of either Embodiment 44 or 45, wherein the disease or disorder is selected from the group consisting of a cancer, an autoimmune disease, a vascular disease, a neurodegenerative disease, a metabolic disease, a renal disease, and an inflammatory bowel disease (IBD).

Embodiment 47. The method of any one of Embodiments 44-46, wherein the EVs are purified using the system of Embodiment 21.

Embodiment 48. The method of any one of Embodiments 44-47, wherein the EVs are purified using the method of Embodiment 22.

Embodiment 49. The method of any one of Embodiments 44-48, wherein the quantifying is by immunofluorescence, western blotting, hybridization, PCR, mass spectrometry, bioanalyzer, nucleic acid sequencing, silver staining, single particle interferometric reflectance imaging sensor (SP-IRIS), or a combination thereof.

Embodiment 50. The method of any one of Embodiments 44-49, wherein the subject is treated for the disease or disorder before step (d).

Embodiment 51. The method of Embodiment 50, wherein: a lower updated expression level of the one or more biomarkers compared to the control expression level indicates that the subject is in remission and/or responding to treatment; a lower updated expression level of the one or more biomarkers compared to the control expression level indicates that the subject is in relapse and/or not responding to treatment; a higher updated expression level of the one or more biomarkers compared to the control expression level indicates that the subject is in remission and/or responding to treatment; or a higher updated expression level of the one or more biomarkers compared to the control expression level indicates that the subject is in relapse and/or not responding to treatment.

### 10. Miscellaneous

The embodiments disclosed herein are only examples, and the scope of this disclosure is not limited to them. Embodiments according to the invention are in particular disclosed in the attached claims directed to a method, a storage medium, a system and a computer program product, wherein any feature mentioned in one claim category, e.g. method, can be claimed in another claim category, e.g. system, as well. The dependencies or references back in the attached claims are chosen for formal reasons only. However, any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

What is claimed is:

1. A multistage dielectrophoretic (DEP) filter system for extracellular vesicles (EVs) purification, comprising:
a plurality of housings, each housing comprising:
an array of fluidic cells, each comprising an input fluid channel and an output fluid channel, configured to receive and process a sample comprising one or more EVs for purification, each fluidic cell comprising:
a semiconductor array comprising a plurality of electrodes;
a plurality of fluid transfer devices in fluid communication between two of the array of fluidic cells via the input fluid channel and the output fluid channels, wherein the fluid transfer devices transfer a processed sample between two fluidic cells via the input fluid channel and the output fluid channel;
an input fluid transfer device in fluid communication with a first fluidic cell in the array, and
an output fluid transfer device in fluid communication with an output fluid channel in a last fluidic cell in the array;
one or more fluid actuators;
one or more storage containers;
at least one electronic control board in operable communication with the plurality of fluid transfer devices, the input fluid transfer device, and the output fluid transfer device; and
a power supply in operable communication with the electronic control board.

2. The multistage DEP filter system of claim 1, further comprising a computer in operable communication with the electronic control board, said computer comprising:
at least one processor;
at least one memory;
an I/O interface;
a display;
at least one communication interface; and
a library of algorithms tangibly stored in the memory and executable by the processor, said algorithms comprising processor-executable instructions to actuate one or more of the plurality of fluid transfer devices, the input fluid transfer device, and the output fluid transfer device.

3. The multistage DEP filter system of claim 1, wherein the semiconductor array in the fluidic cell enables operation of the multistage DEP filter system at an operating mode for EV purification that is selected from one or more of, high capture efficiency, purification, or band-pass.

4. The multistage DEP filter system of claim 3, wherein the band-pass is based on size of the EV.

5. The multistage DEP filter system of claim 1, wherein the electrodes comprise positive electrodes and negative electrodes.

6. The multistage DEP filter system of claim 5, wherein the semiconductor array comprises electrodes disposed in one or more electrode configurations, wherein the configuration enable generation of an electric field for capturing the EVs in the sample.

7. The multistage DEP filter system of claim 6, wherein one or more of the plurality of fluidic cells have an identical electrode configuration (symmetric electrode configuration).

8. The multistage DEP filter system of claim 6, wherein one or more of the plurality of fluidic cells have a non-identical electrode configuration (asymmetric electrode configuration).

9. The multistage DEP filter system of claim 6, wherein the electrodes are disposed in an interdigitated electrode (IDE) geometrical pattern.

10. The multistage DEP filter system of claim 1, wherein the system is constructed out of organic thin films.

11. The multistage DEP filter system of claim 1, wherein one or more of the fluidic cell is coated with an anti-biofouling agent.

12. The multistage DEP filter system of claim 1, wherein the fluidics cell is tunable for one or more EV capture parameters.

13. The multistage DEP filter system of claim 12, wherein the capture parameters are selected from the group consisting of voltage, frequency, field source geometry, fluid medium, solution conductivity, surface coating, electrode pitch and electrode geometry, or a combination thereof.

14. The multistage DEP filter system of claim 1, wherein the fluid transfer device comprises:
a syringe;
an actuator in operable communication with the syringe;
a valve in operable communication with the actuator; and
a motor in operable communication with the valve.

15. The multistage DEP filter system of claim 14, wherein the valve is a solenoid valve.

16. An extracellular vesicle (EV) analysis system, comprising:
a manifold;
a plurality of solenoid actuated valves in fluid communication with the manifold;
a plurality of analyte reservoirs;
one or more analyte isolation and/or tagging chambers;
one or more analyte sensor chambers; and
one or more fluid transfer devices.

17. The analysis system of claim 16, further comprising a housing that houses the multistage DEP filter system.

18. The analysis system of claim 16, wherein the fluid transfer device is a syringe.

19. The analysis system of claim 16, wherein the fluid transfer device comprises:
a syringe;
an actuator in operable communication with the syringe;
a valve in operable communication with the actuator; and
a motor in operable communication with the valve.

20. The analysis system of claim 16, wherein the valve is a solenoid valve.

21. A system comprising:
the multistage dielectrophoretic (DEP) filter system of claim 1;
the EV analysis system of claim 16; and
a computer in operable communication with the multistage dielectrophoretic (DEP) filter system and the detection system, said computer comprising:
at least one processor;
at least one memory;
an I/O interface;
a display;
at least one communication interface; and
a library of algorithms tangibly stored in the memory and executable by the processor, said algorithms comprising processor-executable instructions to operate the multistage dielectrophoretic (DEP) filter system and the detection system.

22. A method for purifying an EV of interest comprising:
(a) obtaining a biological sample comprising EVs;
(b) applying the sample into a first fluidic cell in an array of fluidic cells of a multistage dielectrophoretic (DEP) filter system;
(c) tuning the fluidic cell using one or more capture parameters suitable for capturing the EV of interest from the sample, but not other biological particles;
(d) applying a wash solution into the fluidic cell to move the uncaptured biological particles to a waste reservoir;
(e) stopping the tuning to release the captured EV of interest;
(f) applying an elution solution to the fluidic cell to elute the EV;
(g) applying the eluted EV of interest to a successive fluidic cell in the array;
(h) repeating (c)-(g) n times, where n=total number of fluidic cells in the array-1; and
(i) eluting the EV of interest from the last fluidic cell in the array.

23. The method of claim 22, further comprising analyzing the eluted EV.

24. The method of claim 23, wherein the analyzing is qualitative and/or quantitative.

25. The method of claim 23, wherein the eluted EV is analyzed using immunofluorescence, western blotting, hybridization, PCR, mass spectrometry, bioanalyzer, nucleic acid sequencing, silver staining, single particle interferometric reflectance imaging sensor (SP-IRIS), or a combination thereof.

26. The method of claim 23, wherein the eluted EV is analyzed using an extracellular vesicle (EV) analysis system.

27. The method of claim 23, wherein the eluted EV is analyzed using the extracellular vesicle (EV) analysis system of claim 16.

28. The method of claim 22, further comprising concentrating the eluted EV.

29. The method of claim 22, further comprising storing the eluted EV.

30. The method of claim 22, wherein the EV is purified using the multistage dielectrophoretic (DEP) filter system of claim 1.

31. The method of claim 22, wherein the applying steps are performed using a fluid transfer device selected from a syringe, a pump, and an automated liquid controller.

32. The method of claim 31, wherein the fluid transfer device is a syringe.

33. The method of claim 31, wherein the fluid transfer device comprises:
a syringe;
an actuator in operable communication with the syringe;
a valve in operable communication with the actuator; and
a motor in operable communication with the valve.

34. The method of claim 33, wherein the valve is a solenoid valve.

35. The method of claim 22, wherein the capture parameters are selected from the group consisting of voltage, frequency, field source geometry, fluid medium, solution conductivity, surface coating, electrode pitch and electrode geometry, and a combination thereof.

36. The method of claim 22, wherein the biological sample is plasma, blood, a liquid biopsy, a cell lysate, or a tissue lysate.

37. A method of determining whether a subject is at risk, or suffering from a disease or disorder that needs treatment comprising:
obtaining a sample comprising one or more EVs from the subject;
purifying one or more EVs from the sample;
determining a biomarker expression profile for the purified EVs; and
identifying the subject as at risk, or suffering from the disease or disorder based on the biomarker expression profile.

38. The method of claim 37, further comprising comparing the biomarker expression profile with a reference database of EV biomarker expression profiles, wherein identifying the subject as at risk, or suffering from the disease is based on the comparing.

39. The method of claim 37, further comprising treating the subject.

40. The method of claim 37, wherein the disease or disorder is selected from the group consisting of a cancer, an autoimmune disease, a vascular disease a neurodegenerative disease, a metabolic disease, a renal disease, and an inflammatory bowel disease (IBD).

41. The method of claim 37, wherein the EVs are purified using the system of claim 21.

42. The method of claim 37, wherein the EVs are purified using the method of claim 22.

43. The method of claim 37, wherein the biomarker expression profile is determined using immunofluorescence, western blotting, hybridization, PCR, mass spectrometry, bioanalyzer, nucleic acid sequencing, silver staining, single particle interferometric reflectance imaging sensor (SP-IRIS), or a combination thereof.

44. A method for monitoring a clinical status of a disease or disorder in a subject comprising:
(a) obtaining a control sample comprising one or more EVs from the subject;
(b) purifying from the control sample, EVs associated with the disease or disorder;
(c) quantifying a control expression level of one or more biomarkers associated with the disease or disorder;
(d) obtaining a test sample comprising one or more EVs from the subject;
(e) purifying from the test sample, EVs associated with the disease or disorder; and
(f) quantifying an updated expression level of the one or more biomarkers associated with the disease or disorder, wherein, a change in updated expression level of the one or more biomarkers compared to the control expression level indicates a change in the clinical status of the disease or disorder.

45. The method of claim 44, further comprising repeating steps (d)-(f).

46. The method of claim 44, wherein the disease or disorder is selected from the group consisting of a cancer, an autoimmune disease, a vascular disease, a neurodegenerative disease, a metabolic disease, a renal disease, and an inflammatory bowel disease (IBD).

47. The method of claim 44, wherein the EVs are purified using the system of claim 21.

48. The method of claim 44, wherein the EVs are purified using the method of claim 22.

49. The method of claim 44, wherein the quantifying is by immunofluorescence, western blotting, hybridization, PCR, mass spectrometry, bioanalyzer, nucleic acid sequencing, silver staining, single particle interferometric reflectance imaging sensor (SP-IRIS), or a combination thereof.

50. The method of claim 44, wherein the subject is treated for the disease or disorder before step (d).

51. The method of claim 50, wherein:
a lower updated expression level of the one or more biomarkers compared to the control expression level indicates that the subject is in remission and/or responding to treatment;
a lower updated expression level of the one or more biomarkers compared to the control expression level indicates that the subject is in relapse and/or not responding to treatment;
a higher updated expression level of the one or more biomarkers compared to the control expression level indicates that the subject is in remission and/or responding to treatment; or
a higher updated expression level of the one or more biomarkers compared to the control expression level indicates that the subject is in relapse and/or not responding to treatment.
